(19) United States
(12) Patent Application Publication
(10) Pub. No.: US 2016/0231251 A1
(43) Pub. Date: Aug. 11, 2016

(54) ASSAY TEST DEVICE, KIT AND METHOD OF USING

(71) Applicant: CREDO BIOMEDICAL PTE LTD., Singapore (SG)

(72) Inventors: Chung-Pei OU, Singapore (SG); Kaushal SAGAR, Singapore (SG); Abdur Rub Abdur RAHMAN, Singapore (SG); Winston WONG, JR., Singapore (SG); Stephen Chang-Chi KAO, Singapore (SG); Catia MARQUES, Singapore (SG)

(21) Appl. No.: 14/916,744

(22) PCT Filed: Aug. 28, 2014
(86) PCT No.: PCT/IB2014/002637

§ 371 Date: Mar. 4, 2016

Related U.S. Application Data
(60) Provisional application No. 61/873,463, filed on Sep. 4, 2013.
(60) Provisional application No. 61/919,881, filed on Dec. 23, 2013.

(51) Int. Cl. G01N 21/78; G01N 27/414; G01N 21/80; C12Q 1/70; G01N 33/543; C12Q 1/68; G01N 33/84; G01N 27/416; G01N 33/558

(52) U.S. Cl. CPC G01N 21/78; G01N 27/4167; G01N 27/4145; G01N 21/80; G01N 33/558; G01N 33/5438; C12Q 1/686; G01N 33/84; C12Q 1/703; G01N 2021/752

(57) ABSTRACT

The present invention relates to assay test devices, and methods and kits for use to monitor, sense, read and display results by using devices with printed electronics, such as batteries, reading devices, and other circuitry and/or using colorimetric means for testing by using a sensitive indicator pH dye, or both.

### FIELD OF THE INVENTION

The present invention relates to assay test devices, kits, and methods for using the test devices in order to sense, monitor, differentiate readings and display results the device is meant to test. Previous devices have been available to detect or monitor traces of chemical and/or biological target(s) that exist(s) in a very low concentration in samples, including, not exclusively, biological samples, materials, organic or inorganic samples. The traces of chemical and/or biological target(s) can include biological/chemical products, fragments or whole target(s), e.g. nucleic acid sequences, cells, viruses, pathogens, chemicals, with applications in point of care/site/interest and laboratory in fields such as pharmacogenomics, pathogen detection and monitoring, determination of genetic predisposition, genetic classification for clinical trials, diagnostics, prognostics, infectious disease diagnostics and monitoring, bio-defense, forensic analysis, paternity testing, animal and plant breeding, food testing, human identification, genetically modified organism testing, chemical contamination, food safety, monitoring and tracking in production chain, and production in-line monitoring/control.

This invention is achieved in a combination of ways. One way is through the use of printed electronics in order to sense changes, such as pH, needed to be measured. Another way to measure reaction changes is through the use of colorimetric media (changes in color that are indicated in color changes or that become visible) detected by using printed electronics, as one way to detect the results amongst others. Additionally, printed electronics sensors are used to display measured results on integrated units.

Some devices that exist are, for instance, lateral flow devices and methods for using them in a diagnostic assay. U.S. Ser. No. 14/345,276 incorporated by reference herein in its entirety. The method and devices disclosed in U.S. Ser. No. 14/345,276 are useful for detecting or monitoring targets such as chemical, biological and material targets that are found in very low concentrations in the samples provided. However, the methods and devices of U.S. Ser. No. 14/345,276 do not utilize the methods, devices and kits of the present invention.

In addition to lateral flow devices, micro fluidic fuel cells on paper may be used in the present invention.

Up to this point in time, printed electronics have been used in various technical fields. However, for the first time the display aspect of the devices of the present invention uses printed electronics to display the results being tested. These printed electronics include at least one printed circuit display and/or battery in printed form. It also has been determined that the sensor monitor, differential ready unit and/or display unit is subject to having its electronics in printed form. These electronics such as a circuit, a display and a battery are all located on a medical device such as but not limited to a lateral flow device, in order to read the results intended to be measured by the device.

Printed electronics is printing methods used to create electrical devices on various substrates by using common printing equipment. Patterns are printed on materials, such as screen printing, flexography, gravure, offset lithography, and inkjet because these are typically low cost processes. Electronically functional electronic or optical inks are then used on the substrate, creating active or passive devices, such as thin film transistors or resistors.

The term printed electronics means an organic electronics or plastic electronics, in which one (or more) ink is composed of carbon-based compounds. Printed electronics, in contrast, specifies the process, and, subject to the specific requirements of the printing process selected, can utilize any solution-based material. This includes organic semiconductors, inorganic semiconductors, metallic conductors, and nanoparticles, amongst others.

For the preparation of printed electronics nearly all industrial printing methods are employed.

The most important benefit of printing is the low-cost. The lower cost enables use in more applications. An example is RFID-systems, which enable contactless identification in trade and transport. Also, printing on flexible substrates allows electronics to be placed on curved surfaces.

The present invention further relates to diagnostic, genetic testing, pedigree and breed selection testing, genetic modified organism testing, pathogen detection, genotyping, mutation detection, companion gene testing for prescription or clinical treatment, detection of cancer type, monitoring and prognosis of cancer. Genetic testing has been widely used in clinical application, the food industry, forensic testing, human identification, pathogen epidemic surveillance and detection of new disease strains. This genetic testing covers a range of technologies that involve detection and identification of nucleic acids from analytes. Examples includes DNA sequencing, real-time polymerase chain reaction (PCR), DNA microarray, and restriction fragment length polymorphism (RFLP), as examples. The present invention provides enhanced means by which to carry out such testing, either through the uses of printed electronics or within a system that uses a dye pH indicator, or both.

Traditional methods for detecting nucleic acids that are often times found in minute quantities require multiple devices and steps to process a sample, amplify the target, and detect the amplification. Amplification of nucleic acids, DNA or RNA, has been well established, and there are various methods that exist today for different assay requirements. Even existing reactions methods that monitor nucleotide insertions for PCR that use detected electrical signals do not use printed electronics or the colorimetric methods of the present invention (see U.S. Pat. No. 788,015 B2 and U.S. Pat. No. 8,114,591 B2).

Thermocycling based Polymerase-Chain-Reaction (PCR) based amplification has been shown to be reliable in detecting nucleic acids, as well as gene variations, such as copy number variation or single-nucleotide polymorphism. This method has been well established such that it is often times a standard method for applications that require most regulation such as clinical and forensic applications. Regardless of the nucleic acid amplification methods, the amplified products are not detectable without a visualization method. Current nucleic acid visualization methods relate to attaching a fluorescent probe to the amplification reaction. These probes include the fluorescence tag in a Taqman detection oligo and double-stranded DNA chelator, Sybr Green or other fluorescence chemical that is sensitive to the reaction product. The fluorescence compound is essential in this type of detection because of the high proton emission from the fluorescent molecule, and the emission is only detectable in the presence of the reaction product. The emission only occurs when the fluorescence probe of the Tagman detection oligo is hybridized to the amplified product and cleaved by the DNA polymerase or the Sybr Green is chelated to the amplified product.

However, these fluorescent chemicals are sensitive to light exposure or require special storage conditions such as refrigeration. Exposure to the ambient light causes irreversible damage to the fluorescence chemicals, a phenomenon called photo bleaching. For any fluorescence method, an excitation light source would also be required for any emission to occur. An UV light source is normally used as the excitation light source, to excite the fluorescence probe in order to produce measurable light emission. One example is published by Paul LaBarre of PATH, Seattle, USA (PloS One V6, issue 6, e19738), incorporated in its entirety by reference. Fluorescence emission is possible when the amplification product, pyrophosphate, relieves the fluorescence chemical from being quenching. An UV light source is needed and is provided by a handheld UV LED. The intensity of the light depends on the quantity of the product and the ambient light condition. In the case of comparing an unknown sample to a positive control and a negative control, the single UV LED would not be able to provide uniform illumination to all three samples. It could be hard to differentiate the positive response from a negative one without the help from an instrument. Another source of perturbation is the inconsistent emission from the fluorescence dye. As the emission relies on the swap between two metal ions binding, which is a secondary reaction other than amplification reaction, it is subject to interference from other metal chelators commonly existing in EDTA blood or other operation variations.

Another example where the sample could inhibit or prevent the fluorescence reading is when the solution is not a clear solution, such as when the sample is untreated whole blood. Without precision instruments, it is nearly impossible to handle the sample volume less than 1 micro litre. While most of the nucleic acid reaction is performed under 50 micro litre, more commonly at 25 or 10 micro litre, when the sample is cloudy or strongly coloured, the fluorescence methods are severely restricted. Large dilution or a purification step is required prior to the reaction.

An amplification method for detecting nucleic acids using a pH sensitive system directly measures hydrogen ions rather than using fluorescent dyes. This is accomplished by utilizing CMOS chip technology with an ion-sensitive effect transistor (ISFET) sensor [“A pH-ISFET based micro sensor system on chip using standard CMOS technology,” Haigang Yang et al, Systems-On-Chip for real-time application, Proceeding of the Fifth International Workshop, 2005]. The hydrogen ion sensing layer is the silicon nitride which is the top layer of a CMOS chip. This technology results in cost-effective, nucleic acid analysis. It is essential to be electrically connected for any CMOS chip, and special packaging of the chip is needed to allow the measurement and amplification reaction. As a consequence, the method is expensive and challenging. It is expensive because of the high cost associated with both the design and production of any CMOS chip. It is challenging because of at least two reasons: 1. the risk of short circuit from the amplification liquid leakage via pin hole or minor packaging defect, and 2. The risk of strong interference between the sensing layer, e.g. silicon nitride, and the reaction components. Because of these challenges and concerns, it is not an ideal solution for a genetic test that is cost effective and simple.

The present invention thus also relates to a new method, device and kits that produce a readable electronic set of data or produces color differences in the presence of nucleic acid amplification. For any reaction, it is crucial to keep the reaction container securely sealed after the reaction. This is to prevent other further reactions being contaminated by previously amplified nucleic acid. Any essential component for detection or reaction should be sealed together with the amplification reagents after adding the sample nucleic acid in the reaction. Although it is known that nucleic acid amplification could produce pH drop, it is not known how it could be possible to perform pH depending nucleic acid amplification without the help of instruments, such as checking the right starting pH and performing the necessary adjustment accordingly. It also is not known how it could perform nucleic acid amplification such as a polymerase chain reaction (PCR) without the reaction being inhibited by the extra dye component. For example, in a standard PCR reaction, 10 mM Tris or higher concentrations is always included in the amplification reagents. In the current method, the total buffer capacity could be limited under 5 mM or preferably under 2 mM or preferably under 1 mM such that the pH change will not be inhibited by the buffer.

Another problem that is associated with pH dyes is that the dye inhibits the amplification reaction. The pH indicator dye, such as bromothymol blue at the concentration at 1 mg/mL produces good colour intensity but it inhibits the LAMP reaction completely. When using soluble dye in a nucleic acid amplification method, it is crucial to restrict the contact of the dye and the amplification components. This could be done by dilution or by restricting the molecular contact surface area. By dilution, the concentration of the dye is limited such that the interference is minimal. In a more preferred method, the dye should not be soluble, and the dye exists in a solid phase that is in contact with the amplification reagents such that the molecular contact surface area of the dye to amplification reagents is drastically reduced.

In an amplification, it is possible to provide a stable starting pH, without the presence of extra tris buffer. The colour of the dye from a reaction would not change for many days if the container remains sealed.

Depending on the Mg ion concentration and the starting pH of the amplification, the pH change after the amplification is either positive or negative. Therefore, it is important to control the starting pH. The starting pH is then set by adding a weak buffer component or adding acid/base to adjust. When the pH dye is premixed with the amplification reagents, it is thus easier to know whether the starting pH is right without the need for a pH meter.

Since the dye component could be soluble or exists in the solid surface that is in contact with the reaction, there is no restriction on the form of the reaction chamber as opposite to the ISFET. For visual reading, there should be at least one part of the container that is not completely opaque to allow the viewing. The whole process is compatible with any over-the-shelf reaction vial without the need to design a proprietary of a reaction cartridge, such as the cases in Xpert of Cepheid, or Biofilm Array of BioFire.

### BACKGROUND OF THE INVENTION

Assay tests have been used to analyze test samples, and in particular, lateral flow devices for such uses have been used in point-of-test application since they are easy to use and are relatively inexpensive to use. See U.S. Ser. No. 14/345,276, hereby incorporated by reference in its entirety. These established readable devices are usually dependent on colored particles, such as gold, latex or fluorescence in order to become visible as the analyte comes in contact with the colored particles. This resulting color is viewed by the user. As such, it is possible because there is user interpretation of the colored pattern that there could be inconsistency of how to interpret the results.

In order to help alleviate this potential interpretation of color pattern, digital lateral flow analyzers have been developed in which a separate electronic reader scans and reads the pattern, whether color or fluorescence, on a testing zone of a lateral flow membrane. Examples of these type of meters include ESEQuant Lateral Flow Immunoassay Reader and SNAPshot Dx Analyzer from IDEXX Laboratories.

Some of the types of devices commercially available have built-in issues associated with their use. For instance, a reader-cassette approach makes such devices costly. In most of these situations, a disposable meter would be integrated in the flowing of a lateral flow device. One such device is Clear Blue pregnancy testing device, wherein a colored line at the reaction region is optically sensed to monitor the appearance of a line. This type of device has two lateral flow assay (LFA) strips, a printed circuit board (PCB) with the appropriate electronic components such as photo-optic sensors, processor, LCD (Liquid Crystal Display) and a battery. This type of testing device uses a LFA for hcG, a pregnancy marker. One LFA is the calibration control and the other is the detection strip.

Unfortunately, these types of devices are quite wasteful since the electronic components are disposed of after only one use. Furthermore, the manufacturing of such devices requires multiple steps, thereby increasing costs. Thus, there is need for devices wherein such devices are more easily produced and are not discarded after only one use.

For example, conducting and semi-conducting materials, such as polymers and other molecules are used today in an array of electronics. Such uses include displays in mobile services, with organic electronics (and inorganic electronics) used in such devices instead of traditional electronics.

An example of the use of electronic devices is radio frequency identification (RFID) but the development of a fast switching transistor and antennas of frequencies of 100 KHz plus memory input together is still being sought. Organic transistors may be the solution to providing electronics to surfaces for uses such as test assays.

Adding organic electronics to paper alleviates issues with false readings, counterfeiting, breaching and security issues.

One way in which the present invention overcomes this technology's shortcomings is by utilizing an electronic ink material compatible to both transistor use and other uses. The present invention provides for one mechanism or switch phenomena in use with a material system that modulates charge transport, is used in displays and is used for power storage or conversions, as well.

Since paper is the most produced product made by man, it is a logic substrate to use for organic/inorganic electronics to be used. Paper can reduce the use of a number of pathways and material deposit steps that forms the basis of the present invention.

Printing technologies include sheet-based and roll-to-roll-based printing. Sheet-based inkjet and screen printing are typically used for low-volume, high-precision work. Gravure, offset and flexographic printing are also used for high-volume production. While offset and flexographic printing are mainly used for inorganic and organic conductors, gravure printing is especially suitable for quality-sensitive layers. Organic field-effect transistors and integrated circuits are prepared by means of mass-printing methods.

Screen printing also is used for fabricating electronics due to the ability to produce patterned, thick layers from paste-like materials.

Aerosol Jet Printing is another way to utilize printed electronics. The Aerosol Jet process begins with an atomization of ink, which can be heated up to 80° C., producing droplets on the order of one to two micrometres in diameter. The atomized droplets are entrained in a gas stream and delivered to the print head.

Other methods with similarities to printing, among them microcontact printing and nano-imprint lithography also are useful. Here, μm- and nm-sized layers, respectively, are prepared by methods similar to stamping with soft and hard forms, respectively. Often the actual structures are prepared subtractively, e.g. by deposition of etch masks or by lift-off processes. For example electrodes for OFETs can be prepared.

Both organic and inorganic materials are used for printed electronics. Ink materials must be available in liquid form, for solution, dispersion or suspension and they must function as conductors, semiconductors, dielectrics, or insulators.

Organic printed electronics integrates knowledge and developments from printing, electronics, chemistry, and materials science, especially from organic and polymer chemistry. Organic materials in part differ from conventional electronics in terms of structure, operation and functionality, which influences device and circuit design and optimization as well as fabrication method.

The discovery of conjugated polymers and their development into soluble materials provided the first organic ink materials. Materials from this class of polymers possess conducting, semiconducting, electroluminescent, photovoltaic and other properties.

Organic semiconductors include the conductive polymers poly(3,4-ethylene dioxitiophene), doped with poly(styrene sulfonate), (PEDOT:PSS) and poly(aniline) (PANI). These polymers are commercially available in different formulations and have been printed using inkjet, screen and offset printing or screen, flexo and gravure printing, respectively. The use of a flexible sensor that utilizes a polyalanine layer for sensing pH changes through impedance changes is disclosed in an abstract presented at the Sensing Technology, 2011 Fifth International Conference entitled “Flexible pH sensor with polyaniline layer based on impendance measurement.”

Inorganic electronics provides highly ordered layers and interfaces.

Silver nanoparticles are used with flexo, offset and inkjet. Gold particles are used with inkjet.

Other important substrate criteria are low roughness and suitable wettability, which can be tuned pre-treatment (coating, corona). In contrast to conventional printing, high absorbency is usually disadvantageous.

### SUMMARY OF THE INVENTION

One of the objectives of the present invention is to provide medical testing device assays, devices and kits by utilizing printed electronics wherein the electronic system part of the device is printed by using organic semiconductor materials or inorganic materials.

The present invention provides a functional chemical/organic field transistor as the sensor. Furthermore, the present invention provides a functional printable detecting circuit as the controller, as well as the reader of the result in visable form.

Additionally, the present invention provides a sensor device to measure programmable interval timer (PIT) by using printable components and materials that change electrical conductivity according to PIT levels.

The organic materials useful in the present invention include such materials as polyaniline, poly(3-hexylthiophene), pentacene, polytriarylamine, 5′,5-bis-(7-dodecyl-9H-fluoren-2-yl)-2,2′-bithiopene, polyethylene, naphthalate, and/or poly(4,4′didecylbithiopene-co-2,5-thieno[2,3-b] thiophene). The inorganic materials useful in the present invention include tantanium pentoxide, silver chloride, silver paste, silicon, silicon dioxide, silicon nitride, aluminum oxide and/or other mineral semiconductor, metals and metal oxides. Additionally, nanoparticles, nanotubues and/or graphene are useful in the present invention.

Another object of the present invention is to include transistors, resistors, capacitors, diodes, interconnectors and other pertinent electronics of the invention fabricated by printing methods. Amongst the printing methods that are useful in the present invention are atomic layer deposition, vapour deposition, inject printing, roll-to-roll printing and/or screen printing.

Further, another object of the present invention thus provides a new method for printing these components by utilizing a high volume output system. For example, ink-based roll-to-roll printing can print millions of the electronic components of the present invention, thereby cutting down manufacturing costs and simplifying the overall use of these devices.

Additionally, the present invention has the advantage of being light weight and provides enhanced flexibility to the components. A flexible sensor provides good contact with the lateral flow strip of such devices, but it avoids potential damage to the membrane structure of any such devices. Furthermore, because the actual weight of the printed electronics is much less than a typical printed circuit board (PCB) component, (these include printed circuit boards and discrete components such as packaged logic and memory chips, resistors, inductors, and capacitors) the lateral flow strip is better protected from potential damage.

The present invention integrates sensors into a single system, such as on a sheet. As such, single-use systems can be constructed by integrating the electrochemical transition to suspend to the desired specific substance. Further, any electronic signal then can be amplified and decoded even further to potentially display a numeric value is provided herein.

The present invention therefore comprises a printable electronic sensor system, a transistor, a sensing transistor, controlling circuit, signal processing circuit, display circuit and optionally, battery, printed in a manner that eliminates use of many components.

An embodiment of the present invention is a logic circuit printed to monitor and electrical signal from a sensor over a period of time.

Thus, another embodiment for carrying out methods of the present invention is to a pH indicator method, wherein the sample for example, nucleic acid, amplification flows through a microfluidic channel on a substrate, and as it flows consecutively passes through temperature zones provided in the substrate or base suitable for successive repeats along the length of the channel. The pH indicator dye can be incorporated in all the PCR and other embodiments such as lateral flow device described herein. The observed reading when using this method, device and/or kit is a color change, the appearance of a line or other pattern where no line is found initially, the appearance of a line on a white background or the disappearance of a line to indicate a negative effect, amongst other observations to indicate a reaction result reading.

While the above illustrates generally a PCR system designed to achieve thermo-cycling, various isothermic nucleic acid amplification techniques are known, e.g., single strand displacement amplification (SDA), and DNA or RNA amplification using such techniques may equally be monitored in accordance with the invention.

An object of the present invention is to provide at least one pH indicator dye that is mixed with an amplification reagent before mixing with a sample. When there is an amplification reaction after adding the sample, the pH change causes the color change of the pH indicator dye. The color change is much easier to see by the un-aided eye when the dye is immobilized to a solid matrix, where it is permeable to the hydrogen ion but not DNA polymerase or nucleic acids. Because of differential permeability, it is possible to increase the optical density by increasing they dye concentration in the solid matrix without increasing the risk of inhibiting the reaction. The pH indicator could be the particles or immobilized to the particles. The size of the particles is not limited by the selection of the dye or colour. The size of the particles is only relevant to the choice of the reaction container or condition. The dye could also be immobilized on a film or the surface of the container such that it minimizes the interference to the amplification reaction.

Another objective of the present invention also is to provide a pH sensitive dye used to detect or monitor, for instance, nucleic acid amplification. The dye may be found in solution, on three dimensional objects such as beads or strips, or in a vessel or compartment or combination thereof.

Use of beads may be advantageously used. The beads are spherical particles synthesized from any suitable material for the attachment of the dye, e.g. silica, polystyrene, agarose or dexteran. The particles could be synthesized using a core-shell structural therefore a particle could be form by both paramagnetic materials and a dye hydrogel. The bead from silica, for example, has higher density such that it is easy to keep the bead at a constant position in the solution or moving the bead out of the solution by inverting the reaction vial.

The present invention also has as an advantage use of colorimetric sensing as one way to visualize these assay results through the use of a pH sensitive dye in solution or immobilized on one or more 3D structure such as in bead. Also, the system can take place in a reaction chamber or vessel, while combinations of the above may be used.

Another object of the invention, therefore, is to provide a lateral flow platform that generates a pH or visual signal that can be observed by printed electronic device or colorimetric mechanisms. This objective is achieved in at least three or more combination of ways. One way is through the use of a colorimetric medium (changes color or becomes visible) detected by printed electronic photoconductors. Another way is through printed electronics sensors (changes recorded as the sensor is exposed to different pHs) resulting in different voltage output; and use of a printed electronics sensor (to record change as a sensor is exposed to different pHs) resulting in display of result on integrated display unit(s). Readings are taken at the initial baseline and over a time period. Then, the change is recorded and observation is made of a threshold pH change being affected by the chemical or biological reaction.

In a preferred embodiment of the device of the present invention, the sensor is an ion-sensitive field effect transistor (ISFET). This ISFET is used to sense PIT and monitor pH values. OFET (Organic Field-Effect Transistor) also is useful.

Another object of the present invention is therefore to provide a nucleotide detection platform (quantitative and/or qualitative detection) that generates a pH or visual signal that is observed by a printed electronic device. The amplification method used on this platform is isothermal or PCR, amongst others, and is achieved by the following three methods or more combination:

1) a color metric medium (such as pH dye) (that changes color or becomes visible) is detected by printed electronic photoconductors; 2) Impedance printed electronics sensor (impedance changes as sensor is exposed to different pH.) resulting in different voltage output; and/or 3) Impedance printed electronics sensor (impedance change as sensor is exposed to different pH) resulting in display of result on integrated display unit.

In an embodiment of an ISFET of the present invention, the device has a circuit that is printed to differential measurements in order to monitor what activities are taking place between the control zone, testing zone and background zone. (see FIG. 1).

An optional feature of the present invention uses a hybrid method which comprises a flexible sensor component and ASIC chip that is mounted to the flexible substrate or a printed circuit board.

### BRIEF DESCRIPTION OF THE DRAWINGS

FIG. 1: This figure provides a basic differential mode circuit for measuring the signal difference between a sensor at the testing zone (ISFET1) and the background zone (ISFET2) the control zone is ISFET3.

FIG. 2: A design of the present invention monitoring pH changes at the testing zone.

FIG. 3: An enzyme dependent pH value monitored by a color change.

FIG. 4: A pH indicator of the invention, wherein 1 is sample pad, 2 is conjugate pad, 3 is chromatographic membrane, 4 is test line, 5 is the control line, 6 is absorbent pad and 8 is support material.

FIG. 5A: This figure represents prior systems that utilize individual components.

FIG. 5B: This figure represents the use of material to make the entire system for just a few printing steps.

FIG. 6. The colour of each dye film corresponds to a pH range.

FIG. 7. The photo illustrated the colour response of the pH film in a LAMP reaction for 2C19 genotyping.

FIG. 8. K1 chemical is tested in the form of a film, cellulose particles and soluble molecules.

FIG. 9. The photo shows the colour of the dye in each tube prior to the LAMP reaction.

FIG. 10. This photo shows the colour of the dye change in the tube where amplification occurs in the LAMP reaction in the top row while the colour of the dye is unchanged where there is not amplification in the LAMP reaction in the bottom row.

FIGS. 11A and B. This shows two distinct films for amplification detection testing.

FIG. 12. Bromothysial blue does not produce a colour change, and the pH remains unchanged.

FIG. 13. This figure illustrates the example sensor and the pH test results.

FIG. 14. The dye colour of each tube is pink prior to the LAMP reaction.

FIG. 15. The dye colour then changes to yellow for tubes 1 to 7 and remains pink for tubes 8-10.

FIG. 16. This chart shows the positive and negative discrimination response.

FIG. 17. These are agarose electrophoresis photos showing the LAMP amplification in lanes 1 to 7.

FIG. 18. This shows the dye colour prior to the reactions that are positive or negative with regard to DNA.

FIG. 19. This shows the dye colour after to the reactions that are positive or negative with regard to DNA.

FIG. 20. This is a whole blood effect on dye colour prior to the reaction.

FIG. 21. This is a whole blood effect after the reaction.

FIG. 22. This shows the colour of the immobilized dye after shaking the solution off the dye.

FIG. 23. This is s LAMP reaction from each tube using agarose electrophoresis.

FIG. 24. This shows the result of a PCR reaction with the presence of dye.

FIG. 25. This is a schematic of the physical entrapment and chemical linkage pH indicator dye to the cross-linked polymer matrix.

FIG. 26. This shows the colour difference between the reaction versus no reaction when hydrogel slabs are used.

FIG. 27. This shows an example of a pH responsive dye conjugated hydrogel of polyurethane on a cellulose acetate ball of 2 mm diameter.

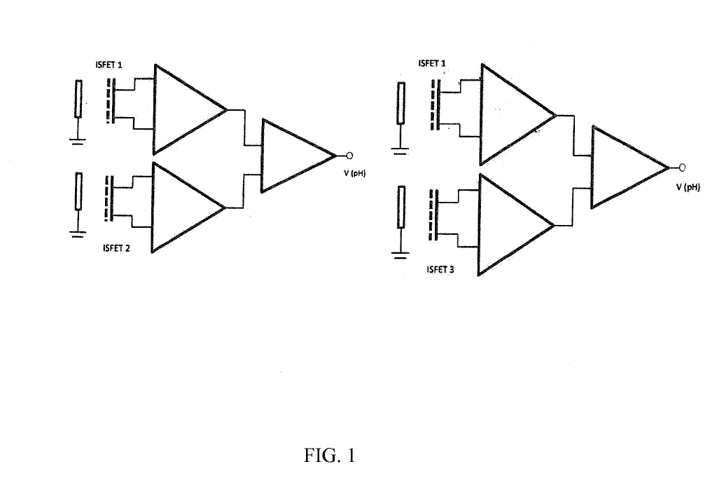

### DETAILED DESCRIPTION OF THE INVENTION

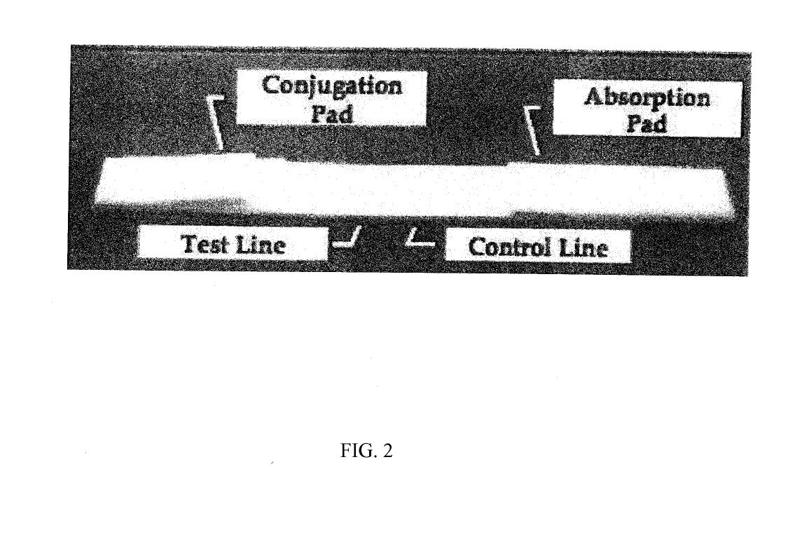

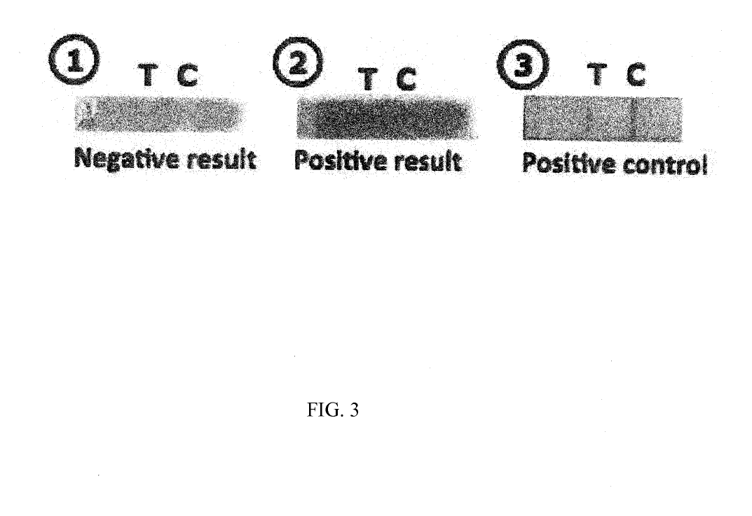

One of the devices of the present invention is comprised of a conjugation pad, absorption pad, test line and control line, as illustrated in FIG. 2. When a sample is loaded onto the conjugation pad, due to papillary forces, that sample moves toward the Test Line. A reagent is in the conjugation pad (this may be a sample collection tube) In the middle, the two are separate by a printed monitor at the Test Line and Control Line. When there is analyte present, an immunocomplex is formed at the Test Line. The Control Line indicates active reagents being present. Optionally, the strip may comprise a chromatographic membrane.

Printing of electronics is well documented, but not in a diagnostic devices. Some of the technologies used for printing of electronics, include organic LED displays, flexible touch screens, RFID super capacitors and photo voltaic membranes. Organic LED used as the display portion can show a Yes/No answer with different colors or fonts.

Smart packaging also can be used as OLED. A device of the present invention shows a preset message using OLED.

RFID can be used in an inventory control and counterfeit prevention way. The integration of printed electronics allows for anti counterfeiting (For instance, validation of a device serial code against local/remote databases to make sure that the device has never been used or is not stole nor is not used at a location where the device is not approved can be accomplished by the present invention). In this invention, that device knows what target it must detect, and it displays the target with the result.

As an example, the interconnectors on printed electronics can use silver, used as a conductive ink. The circuit of the present invention uses conductive inks, semiconductor inks, doping and dielectric substances including the divider, comparator, NOT gate and amplifier.

All primers used in the present examples are synthesized by Integrated DNA Technologies or Thermo Fisher. As the presence of the pH dye in the reaction causes minimal effect on the amplification reaction, there is no need to change the composition of the amplification reagents. The only exception is that the magnesium ion (Mg2+) should be high enough, e.g. 1.5 mM or preferably 2 mM or higher, such that deoxynucleotides form complexes with the magnesium ion.

LAMP is a process of amplification of double-stranded DNA that use primers in order to hybridize to the DNA and in order to target a specific sequence of interest. The amplification is achieved by primers forming hybridization with the template DNA extension from the inner primer which is later replaced by an outer primer by the strand-displacement activity of the polymerase and the exponential amplification of the target sequence and the newly synthesized strands.

The primer, deoxynucleotides (dNTPs), reaction buffer, indicator dye, and polymerase are premixed without particular order of the step, apart from the polymerase which is added in the last step to prevent non-specific reaction. For reactions that use non-lyophilised formulation, the reagents above should be assembled on a chilled box to prevent non-specific reaction. The sample DNA, such as purified human genomic DNA, animal DNA, plant DNA, fresh human whole blood, lambda DNA, pUC19 plasmid, different viruses, any nucleic acid segments, either naturally occurring or synthetic, or chimeric, artificial nucleic acid analogues, such as peptide nucleic acids (PNA), morpholino and locked nucleic acids (LNA), asglycol nucleic acids (GNA), threose nucleic acids (TNA) and synthetic bases, or any other nucleic acid template such as RNA is added at the last step before sealing the container and putting the container to a heat block, if heating is required. At the end point of the amplification reaction, the reaction container is observed by the un-aided eye or by a simple camera.

Other targets that form the present invention include, but are not limited to, amino acid sequences of lipoproteins such as peptides, polypeptides, glycoproteins, lipoproteins, such as alpha-fetoprotein (AFP), prostate-specific antigen (PSA), amyloid beta and HIVp24 protein.

Additionally, saccharide polymers such as bacterial capsular polysaccharides and lipopolysaccharide such as endotoxin are used in the present invention.

Specific viruses and/or bacterial diseases that are diagnosed using the methods, devices and kits of the present invention include but are not limited to hepatites B (HBV), hepatites C (HCV), herpes virus, HIV, human papilloma virus (HPV), Ebola virus, white spot syndrome on shrimp, feline leukemia virus, amongst others. The proteins that are associated with diagnosis of these and form the device, kits and methods of the present invention include recombinant nucleoprotein, glycoprotein of Zaire Ebola virus, and S-gene proteins also; hepatites B core multiepitopes; anti-HCV immunoglobulin G and recombinant B virus glycoproteins.

Bacterial diseases that are diagnosed by the present invention include syphilis, chlamydia and gonorrhea.

When lyophilised reagent is used, the first step is to re-suspend the dried reagent with water before adding the sample target. The rest of the steps follow the same order described in the non-lyophilised reaction.

The present examples also provide a device, kit and method for measuring pH changes by the use of a colorimetric method measuring and electronic printing method.

Fabricated nanoliter reactor chambers in silicon with integrated actuators (heaters) for PCR monitoring exist, see for example Iordanov et al. Sensorised nanoliter reactor chamber for DNA multiplication, IEEE (2004) 229-232 (incorporated herein by reference in its entirety) exist. As noted by I ordanov et al. in the above-noted paper, untreated silicon and standard silicon-related materials are inhibitors of Taq polymerase. Therefore, when silicon or a silicon-related material, e.g. silicon germanium or stained silicon (hereinafter “silicon”) is employed for fabrication of the chamber or channel for nucleic acid amplification, it will usually be covered with material to prevent reduction of polymerase efficiency by the silicon, such as SU8, polymethyl-methacrylate (PMMA), Perspex™ or glass.

Microfabricated silicon-glass chips for PCR are also described by Shoffner et al. In Nucleic Acid Res. (1996) 24, 375-379 incorporated herein by reference in its entirety. Silicon chips are fabricated using standard photolithographic procedures and etched to a depth of 115 μm. Pyrex™ glass covers are placed on top of each silicon chip and the silicon and glass are bonded. These are but a few examples of surfaces for use in the present invention. Others include oxidized silicon.

As an alternative, the sample for PCR monitoring may flow through a channel or chamber of a microfluidic device. Thus, for example, the sample may flow through a channel or chamber which passes consecutively through different temperature zones suitable for the PCR stages of denaturing, primer annealing and primer extension.

Thus, in one embodiment for carrying out the present method, the sample for nucleic acid amplification flows through a microfluidic channel on a substrate, and as it flows consecutively passes through temperature zones provided in the substrate or base suitable for successive repeats along the length of the channel. The pH indicator dye can be incorporated in all the PCR embodiment described herein above.

While the above illustrates generally a PCR system designed to achieve thermo-cycling, various isothermic nucleic acid amplification techniques are known, e.g., single strand displacement amplification (SDA), and DNA or RNA amplification using such techniques may equally be monitored in accordance with the invention.

LAMP is a process of amplification of double-stranded DNA that use primers in order to hybridize to the DNA and in order to target a specific sequence of interest. The amplification is achieved by primers forming hybridization with the template DNA extension from the inner primer which is later replaced by an outer primer by the strand-displacement activity of the polymerase and the exponential amplification of the target sequence and the newly synthesized strands.

The following examples are provided as illustrative of the present invention and are not limitative thereof.

### Example 1

A pH detection device and method of using that device, has pH sensitive sensor, controlling circuitry for calculating signal strength and display pixels all using roll-to-roll printing or screen printing of the electronics of the device. These are printed on dielectric materials, such as flexible plastic. The production of the device avoids waste due to device damage and is produced in high production volume. This device is useful as an LFA wherein line observation is indicative of the reaction result.

### Example 2

The printed electronics of Example 1 are placed atop of the LFA strip, with the pH sensitive sensor, controlling circuit (that calculates the signal strength) and display pixels all printed via roll-to-roll or screen printing. These electronic components are printed onto a dielectric material such as a flexible plastic.

In particular, in order to measure pH, the printed electronic system is in contact with a chromatographic media. The changes in pH are monitored, as is the rate of the pH change by measuring at 10 minutes and at 30 minutes. Glue, amongst others, may be added as a mediator layer.

### Example 3

The printed sensor also includes a printed battery, or as an optional feature, a button battery to power the circuit. The choice of the battery material should not have fire hazard or chemical hazard properties. Preferably, the battery includes a self-test function. The printed sensor contains a display monitor (e.g. OLED Organic Light Emitting Diode) to indicate the result (Yes/No/failure, etc.). This printed sensor has the capability to do simple calculation. As an optional element, the sensor has a communication module (e.g. RFID) to upload the patient information and result to a base station (such as a laptop PC, customized base station, touchpad. Smartphone or combinations thereof).

Another optional element is a sensor that is able to download the patient information from a base station (e.g. laptop PC, customized mobile unit, touchpad, smartphone or combinations thereof) such that the particular device is ‘burned’ with a patient's ID. These communications are optionally encrypted into either a wired system or wireless system.

All of the sensor components are integrated in a continuous printing production process (e.g. roll-to-roll). The assembly of the sensor and the LFA are separately made. Additionally, a temperature sensor is present to compensate/adjust/prevent the use of the device, if the ambient temperature is not within the determined acceptable range.

Fabricated nanoliter reactor chambers in silicon with integrated actuators (heaters) for PCR monitoring exist, see for example Iordanov et al. ‘Sensorised nanoliter reactor chamber for DNA multiplication, IEEE (2004) 229-232 (incorporated herein by reference in its entirety). As noted by I ordanov et al. in the above-noted paper, untreated silicon and standard silicon-related materials are inhibitors of Taq polymerase. Therefore, when silicon or a silicon-related material, e.g. silicon germanium or stained silicon (hereinafter “silicon”) is employed for fabrication of the chamber or channel for nucleic acid amplification, it will usually be covered with material to prevent reduction of polymerase efficiency by the silicon, such as SU8, polymethyl-methacrylate (PMMA), Perspex™ or glass.

Microfabricated silicon-glass chips for PCR are also described by Shoffner et al. In Nucleic Acid Res. (1996) 24, 375-379 incorporated herein by reference in its entirety. Silicon chips are fabricated using standard photolithographic procedures and etched to a depth of 115 μm. Pyrex™ glass covers are placed on top of each silicon chip and the silicon and glass are bonded. These are but a few examples of surfaces for use in the present invention. Others include oxidized silicon.

As an alternative, the sample for PCR monitoring may flow through a channel or chamber of a microfluidic device. Thus, for example, the sample may flow through a channel or chamber which passes consecutively through different temperature zones suitable for the PCR stages of denaturing, primer annealing and primer extension.

Thus, in one embodiment for carrying out the present method, the sample for nucleic acid amplification flows through a microfluidic channel on a substrate, and as it flows consecutively passes through temperature zones provided in the substrate or base suitable for successive repeats along the length of the channel. The pH indicator dye can be incorporated in all the PCR embodiment described herein above.

While the above illustrates generally a PCR system designed to achieve thermo-cycling, various isothermic nucleic acid amplification techniques are known, e.g., single strand displacement amplification (SDA), and DNA or RNA amplification using such techniques may equally be monitored in accordance with the invention.

All primers used in the present examples are synthesized by Integrated DNA Technologies or Thermo Fisher. As the presence of the pH dye in the reaction causes minimal effect on the amplification reaction, there is no need to change the composition of the amplification reagents. The only exception is that the magnesium ion (Mg2+) should be high enough, e.g. 1.5 mM or preferably 2 mM or higher, such that deoxynucleotides form complexes with the magnesium ion.

LAMP is a process of amplification of double-stranded DNA that use primers in order to hybridize to the DNA and in order to target a specific sequence of interest. The amplification is achieved by primers forming hybridization with the template DNA extension from the inner primer which is later replaced by an outer primer by the strand-displacement activity of the polymerase and the exponential amplification of the target sequence and the newly synthesized strands.

The primer, deoxynucleotides (dNTPs), reaction buffer, indicator dye, and polymerase are premixed without particular order of the step, apart from the polymerase which is added in the last step to prevent non-specific reaction. For reactions that use non-lyophilised formulation, the reagents above should be assembled on a chilled box to prevent non-specific reaction. The sample DNA, such as purified human genomic DNA, fresh human whole blood, lambda DNA, pUC19 plasmid, or any other nucleic acid template is added at the last step before sealing the container and putting the container to a heat block, if heating is required. At the end point of the amplification reaction, the reaction container is observed by the un-aided eye or by a simple camera.

When lyophilised reagent is used, the first step is to re-suspend the dried reagent with water before adding the sample target. The rest of the steps follow the same order described in the non-lyophilised reaction.

### Example 4

Similar to inorganic field-effect transistors (FET), an OFET has a source, drain and gate electrodes. The channel current between the source and the drain electrodes is modulated by the gate voltage due to the field effect doping. Compared with silicon FETs, OFETs show relatively lower carrier mobility and stability but better performance in terms of flexibility, biocompatibility, large area and solution processibility. Therefore OFETs are suitable for the applications in disposable sensors.

Table 1 summarizes examples of OFET-based chemical and biological sensors.

TABLE 1
summarises examples of OFET-based
chemical and biological sensors.

| Sensor | | Active | Detection | | |
|---|---|---|---|---|---|
| type | Analyte | layer | limit | Voltage | Reference |
| Ion | K+ | P₃HT | 33 mM | 29 μA/mM | Ji et al., |
| sensor | | | | | 2008 |
| | Na+, | P₃HT | 0.001% | 1.4 × 10−4 A | Scarpa et al., |
| | K+, | | | for Ca2+, | 2010 |
| | Ca2+ | | | 2.2 × 10−6 A | |
| | | | | K+, Na+ | |
| | | | | 1.3 × 10−6 | |
| | SO42− | PDTT | 1 mM | −1.7q per | Madalena et |
| | | | | protein | al., 2010 |
| pH | pH | P₃HT | 6.6-9.5 | 0.071 μA/pH | Ji et al., |
| sensor | | | | | 2008 |
| | pH | P₃HT | 4-10 | — | Scarpa et al., |
| | | | | | 2010 |
| | pH | Pentacene | 4-10 | — | Loi et al., |
| | | | | | 2005 |
| | pH | Pentacene | 2.5-7 | — | Carboni et |
| | | | | | al., 2009 |
| | pH | | | | |
| DNA | | | | | |
| sensor | | | | | |
| Protein | | | | | |

### Example 5

An example of a diagnostic kit of the present invention contains a lateral flow assay device that comprises a chromatographic medium. This chromatographic medium includes: (a) a sample loading zone located upstream of a detection zone; (b) a reporting carrier zone located between the sample loading zone and a detection zone, wherein the reporting carrier zone comprises a reporting carrier capable of forming a complex with the analyte. The reporting carrier of the invention comprises a carrier and one or more proficient enzyme cassettes. Proficient enzyme cassettes or proficient enzyme are defined as an assembly of enzyme(s) which catalyze a reaction such that the rate is more than 1000 per second per enzyme cassette. The value is also called turnover rate or Kcat in enzymology; and (c) a detection zone, wherein the detection zone comprises a capture component for the analyte and an indicator. The sample is contacted in the application zone with the test sample, wherein the test sample travels through the reporting carrier zone along the chromatographic medium from the sample loading zone to the detection zone and beyond the detection zone. Adding a substrate to the detection zone wherein the substrate undergoes a reaction in the presence of a proficient enzyme analyte containing a reporting carrier and generating a response of the indicator within the detection zone that corresponds to the presence or absence of the analyte in the test sample is accomplished with this device.

### Example 6

An example of a diagnostic kit of the present invention detects the presence or absence of an analyte using an enzyme-aided amplification method in a chromatographic medium. The analyte is a biomarker such as an antigen that could be recognized by an antibody or antibody-similars. In this example, the reporting carrier has a first antibody which then binds to the analyte and proficient enzyme. The capture component comprises a second antibody which binds to the analyte at a different epitope from the first antibody. In one embodiment, the first antibody is covalently cross-lined to the proficient enzyme.

The reporting carrier comprises streptavidin and biotinylated proficient enzyme and biotinylated antibody. The first antibody is bound or associated with the proficient enzyme through non-covalent streptavidin-biotin interaction.

### Example 7

In another example of the present invention, the analyte is a sequence of nucleic acids. In this example the reporting carrier comprises a first sequence of nucleic acids which hybridise to one part of the target nucleic acid sequence that is a proficient enzyme which is associated with the first nucleic acid. The first nucleic acid is covalently cross-lined to the proficient enzyme. The reporting carrier comprises streptavidin and biotinylated proficient enzyme and biotinylated first nucleic acid. The first nucleic acid is associated to the proficient enzyme through non-covalent Streptavidin-Biotin interaction.

The reporting carrier binds to p24 protein of HIV in one example of the present invention. In another application, the reporting carrier binds to HIV nucleic acid. The substrate is a liquid solution as part of the device. The solution will be added onto the strip in the last stage.

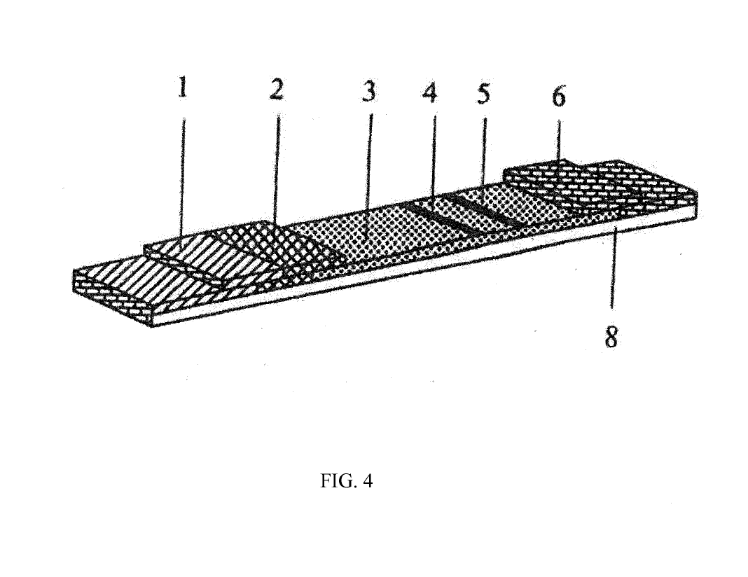

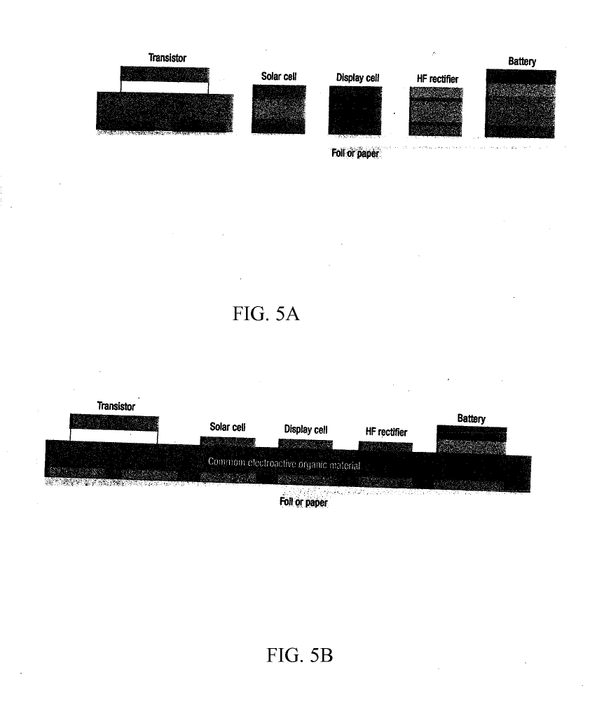

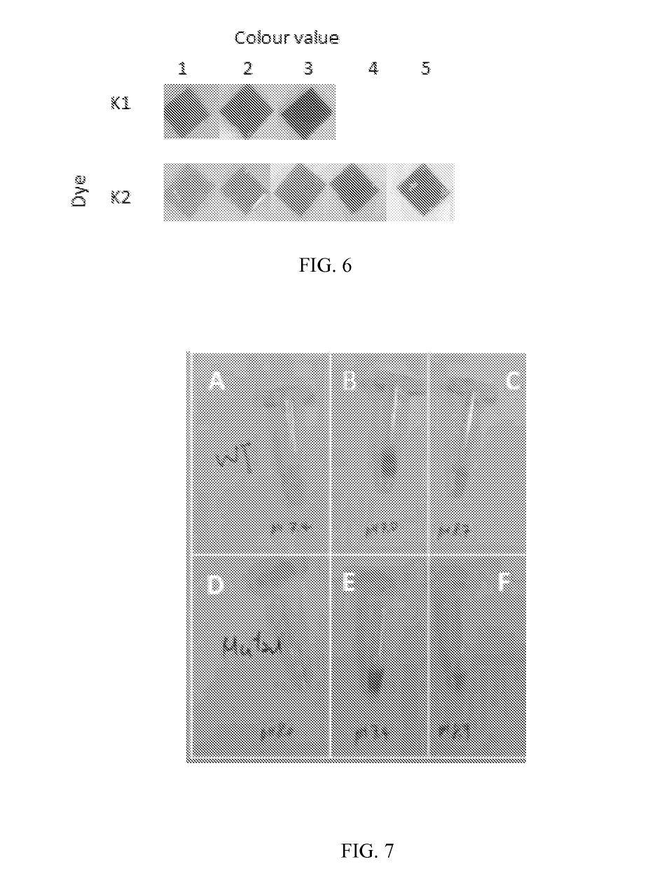

In one embodiment, the test line (4 in FIG. 4) also comprises the pH color indicator. The color of the line (4 in FIG. 4) changes in the presence of the analyte. There is also the same pH color indicator on the C line (5 in FIG. 4). The color changes in the presence of the reporting carrier. Example of the embodiment is found in FIG. 11.

In one embodiment, the pH color indicator is a film, which is placed on top of the test and control lines. In another embodiment, the pH color indicator is printed into the chromatographic medium at the position of the Test and Control lines.

In another embodiment, the substrate solution contains the pH indictor. The color of the test line (4 in FIG. 4) appears in the presence of the analyte due to pH change. The color of the control line (5 in FIG. 4) changes in the presence of the reporting carrier. Example of the response is also shown in FIG. 2.

In another embodiment, the target is the core protein of human hepatitis C virus. The first antibody specific to the first epitope of the core protein is linked to a reporting carrier. A second antibody specific to the second epitope of the core protein is immobilized on the detection zone of the chromatographic medium. The disclosed method detects at least 0.5 pg of protein or better. The proteins include virus proteins such as but not limited to HIV, hepatitis B (HBV), hepatitis C (HBV), human papilloma virus (HPV), Ebola virus, herpes virus, as examples; oncology proteins (prostate-specific antigen (PSA) for prostate cancer; cancer antigen 125 (CA 125) for ovarian cancer; calcitonin for medullary thyroid cancer; alpha-fetoprotein (AFP) for liver cancer; and human chorionic gonadotropin (HCG) for germ cell tumors, such as testicular cancer and ovarian cancer); cardiovascular proteins, such as human cardiac troponin T or I, or pulmanary proteins, hepatic proteins, renal proteins and neurological proteins, such as amyloid beta protein; bacterial proteins such as those used to detect syphilis, chlamyda and gonorrhea.

In another embodiment, the target is the human cardiac troponin T and/or cardiac or/and troponin I for myocardial infarction. The diagnostic kit comprises the printed electronic sensor and controlling circuit to provide quantitative results for rapid measurements. The sensitivity of the kit is also used for high-sensitivity cardiac troponin I assays.

In another embodiment, the electrical sensors produce continuous pH reading over a period of time to produce a time-dependent pH curve. The Yes/No answer of the detection looks at the threshold value by comparing the curves of the different testing zone, control zone, and the any other point on the chromatographic media except the testing zone or control zone. Similarly, a semi quantitative or a quantitative measurement is performed by comparing the curves of the testing zone, control zone, and another point on the chromatographic media except the testing/control zone. Additionally, differential readings of the reaction are also recorded when monitoring and recording the progress of the reaction over time.

### Example 8

The K1 film is a cellulose film of 20 micrometer thickness conjugated with potassium 1-hydroxyl-4-[4-(hydroxyethylsulphonyl)-phenylazo]-naphthalene-2-sulphonate.

The K2 film is a cellulose film of 20 micrometer thickness conjugated with 4-[4-(2-hydroxylethanesulfonyl)-phenylazo]-2,6-dimethoxyphenol.

The K1 solution is potassium 1-hydroxyl-4-[4-(hydroxyethylsulphonyl)-phenylazo]-naphthalene-2-sulphonate.

The K1 particles are Cellulose Microparticles Avicel® PH-101. 50 micrometer in diameter is conjugated with potassium 1-hydroxyl-4-[4-(hydroxyethylsulphonyl)-phenylazo]-naphthalene-2-sulphonate.

### Detection Using Different Dye Forms:

Three different forms of K1 dye are used in the assay, K1 film, K1 particle, and soluble K1. The assay shows the compatibility of the dye form and the LAMP reaction. The LAMP reactions are set up to use p450 2C19 wild type primer set and K562 genomic DNA. 1 ng of K562 which is about 300 copies is mixed with the reaction components. Dye is included in each tube before the reaction. The reaction is held at 63 degree Celsius for 30 mins, and the colour of the reaction is observed.

| | Final concentration | |
|---|---|---|
| Primers mix solution | | |
| 2C19_FIP.Wild | 1.6 | uM |
| 2C19_BIP.Wild | 1.6 | uM |
| 2C19_LF | 0.8 | uM |
| 2C19_LB | 0.8 | uM |
| 2C19_F3 | 0.2 | uM |
| 2C19_B3 | 0.2 | uM |
| Mutant primers mix solution | | |
| 2C19_FIP.Mut | 1.6 | uM |
| 2C19_BIP.Mut | 1.6 | uM |
| 2C19_LF | 0.8 | uM |
| 2C19_LB | 0.8 | uM |
| 2C19_F3 | 0.2 | uM |
| 2C19_B3 | 0.2 | uM |
| LAMP buffer | | |
| KCl | 50 | mM |
| MgSO4 | 5 | mM |
| NH₄Cl | 5 | mM |
| BSA | 1 | mg/mL |
| Tween 20 | 0.10% | |
| Betain | 1M | |
| Deoxynucleotides | 2.8 | mM |
| Bst polymerase | 32 | U |

| | |
|---|---|
| H2O | Fill to 50 uL |

The photo shows the colour response of the pH film in LAMP reaction for 2C19 genotyping. The photo is taken after the LAMP reaction. In the graph the order are K1 film wildtype (A) or mutant (D); K1 powder wildtype (B) or mutant (E); K1 solution wildtype (C) or mutant (F).

Table 2 summarizes the colour value and the pH value of the K1 dye in the LAMP reaction that are provided.

| | | Before reaction | | After reaction | |
|---|---|---|---|---|---|
| | | pH | Colour | pH | Colour |
| | | value | value | value | value |
| Positive control | No dye | 8.7 | 0 | 6.4 | 0 |
| No template control | No dye | 8.7 | 0 | 7.8 | 0 |
| 2C19 Wildtype | K1 Film | 8.7 | 3 | 7 | 1 |
| | K1 Powder | 8.7 | 3 | 7.4 | 1 |
| | (1 mg) | | | | |
| | Soluble K1 | 8.7 | 3 | 8.7 | 3 |
| 2C19 Mutant | K1 Film | 8.7 | 3 | 7.4 | 1 |
| | K1 Powder | 8.7 | 3 | 8 | 3 |
| | (1 mg) | | | | |
| | Soluble K1 | 8.7 | 3 | 8.7 | 3 |

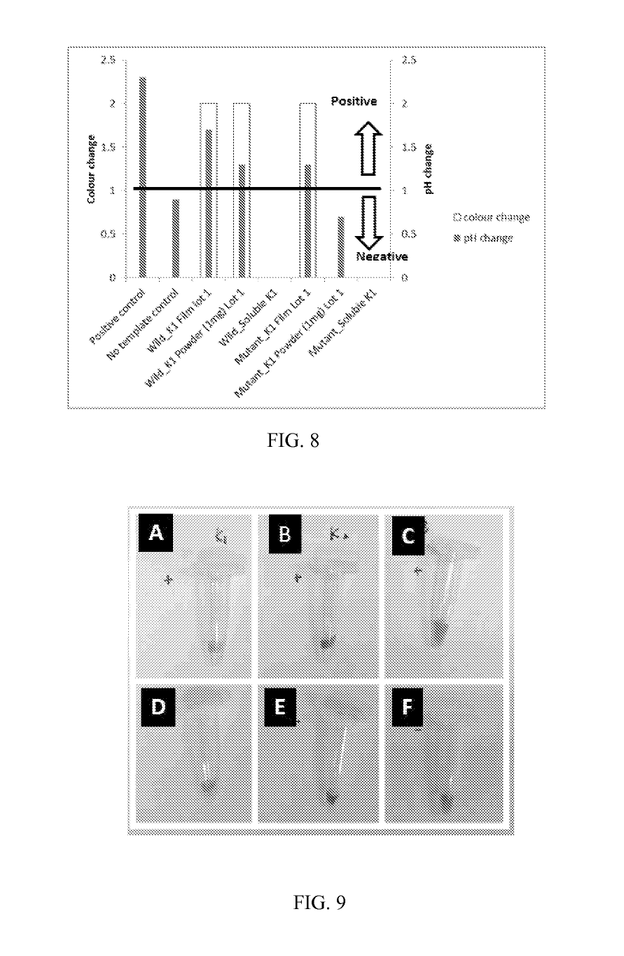

The K1 chemical is tested in the form of film, cellulose particles, and soluble molecules. (See above graph in FIG. 8). The colour change of the 2C19 genotyping is converted into numbers using the colour panel. The chart shows the pH value change (Starting pH-end pH) and the colour change (starting colour-end-colour). When the threshold is held at 1 for colour change or for pH change, the sample with LAMP reaction is distinct from the one without the LAMP reaction. The pH value change is 100% in agreement with the colour change.

### Example 9

The reactions are set up to use p450 2C19 wild type primer set and K562 genomic DNA. 1 ng of K562 which is about 300 copies mixed with these reaction components. pH indicator dye is included in each tube before the reaction. The dNTPs is replaced by a 2.8 mM mixture of (deoxyadenosine triphosphate, deoxyguanosine triphosphate, deoxycytidine triphosphate) in the negative control samples. The reaction is held at 63 degree Celsius for 30 mins and the colour of reaction is observed.

| | Final concentration | |
|---|---|---|
| Wild type primers mix solution | | |
| 2C19_FIP.Wild | 1.6 | uM |
| 2C19_BIP.Wild | 1.6 | uM |
| 2C19_LF | 0.8 | uM |
| 2C19_LB | 0.8 | uM |
| 2C19_F3 | 0.2 | uM |
| 2C19_B3 | 0.2 | uM |
| LAMP buffer | | |
| KCl | 50 | mM |
| MgSO4 | 5 | mM |
| NH₄Cl | 5 | mM |
| BSA | 1 | mg/mL |
| Tween 20 | 0.10% | |
| Betain | 1M | |
| Deoxynucleotides | 2.8 | mM |
| Bst polymerase | 32 | U |

| | |
|---|---|
| H2O | Fill to 50 uL |

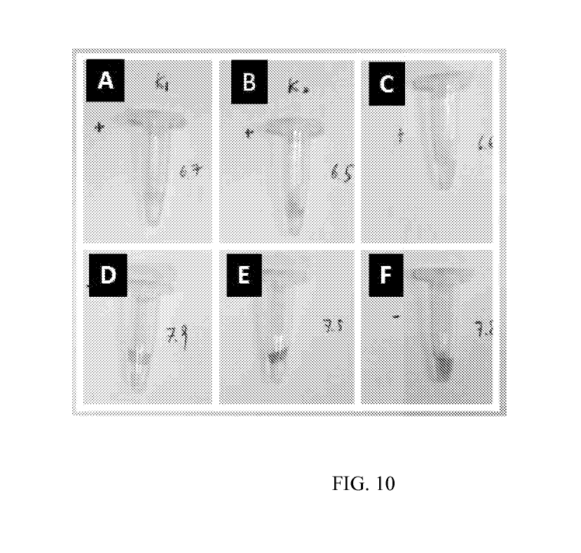

In each tube, a distinct dye film (K1 and K2) or a soluble pH indicator (bromothymol blue, 0.1 mg/mL) is mixed with the amplification reagents before the LAMP reaction. Two distinct films are tested for amplification detection. The photos of the reaction set up and results are shown in FIGS. 9 and 10. The colour changes are converted into values by use of the coding panel. The compiled values are shown in Table 3. To visualize the colour difference and amplification versus no amplification, the value are plotted in FIG. 8.

The result shows colour changes in the presence of the template.

The photo shows the colour of the dye in each tube before the LAMP reaction. In the photo the tubes are K1 film LAMP reaction with template (A) and without template (D), K2 film with template (B) and without template (E), bromothymol blue solution with template (C) and without template (F).

FIGS. 9 and 10 show the colour of the dye changes in the tube where amplification occurs in the LAMP reaction (top row) while the colour of the dye remains unchanged where there is no amplification in the LAMP reaction (bottom row). In the photo the tubes are K1 film

LAMP reaction with template (A) and without template (D), K2 film with template (B) and without template (E), bromothymol blue solution with template (C) and without template (F) (See FIG. 10).

Table 3 presents colour results and the pH correlation to the LAMP reaction.

| | | Before | | After | |
|---|---|---|---|---|---|
| | | LAMP | | LAMP | |
| | Lamp | pH | Colour | pH | Colour |
| | reaction | value | value | Value | value |
| K-1 | Yes | 8.7 | 3 | 6.7 | 1 |
| | No | 8.7 | 3 | 7.9 | 3 |
| K-2 | Yes | 8.7 | 5 | 6.5 | 1 |
| | No | 8.7 | 5 | 7.5 | 3 |
| BB | Yes | 8.7 | Blue | 6.6 | Blue |
| | No | 8.7 | Yellow | 7.8 | Yellow |

Two distinct films are tested for amplification detection. Two distinct pH indicators are immobilised on cellulose films. The colour change of each film is converted into a number using its own colour panel. Table 3 shows the pH value change (Starting pH-end pH) and the colour change (starting colour-end colour). The value of LAMP reactions is distinctly differentiate from the one without the LAMP reactions in all three dye films. The pH value change is 100% in agreement with the colour change. The sample from each tube is analysed using agarose electrophoresis in FIG. 12.

The intensity of the colour change is very strong such that the result could easily be determined by the un-aided eyes. Significant colour change is also present when a soluble dye (bromothymol blue, 0.1 mg/mL) is used as an indicator. It shows that it is possible to use soluble dye.

However, at higher concentration, the dye inhibits the reaction. A similarity is also observed when a soluble K1 dye is mixed with the LAMP reaction. The soluble chemical is prone to interfere and inhibit the amplification.

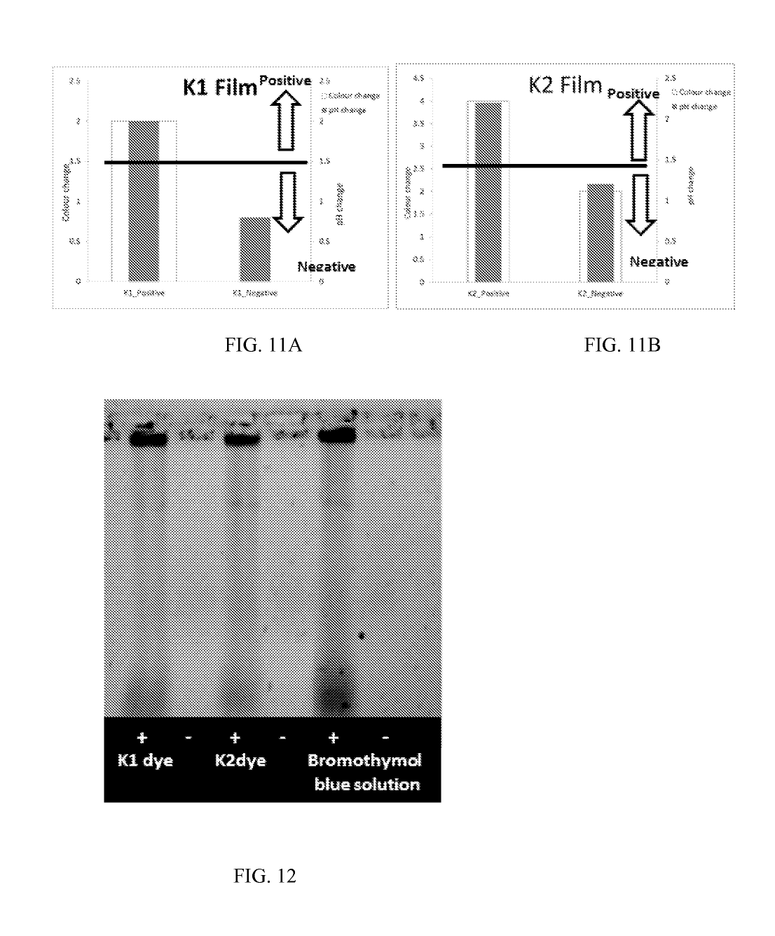

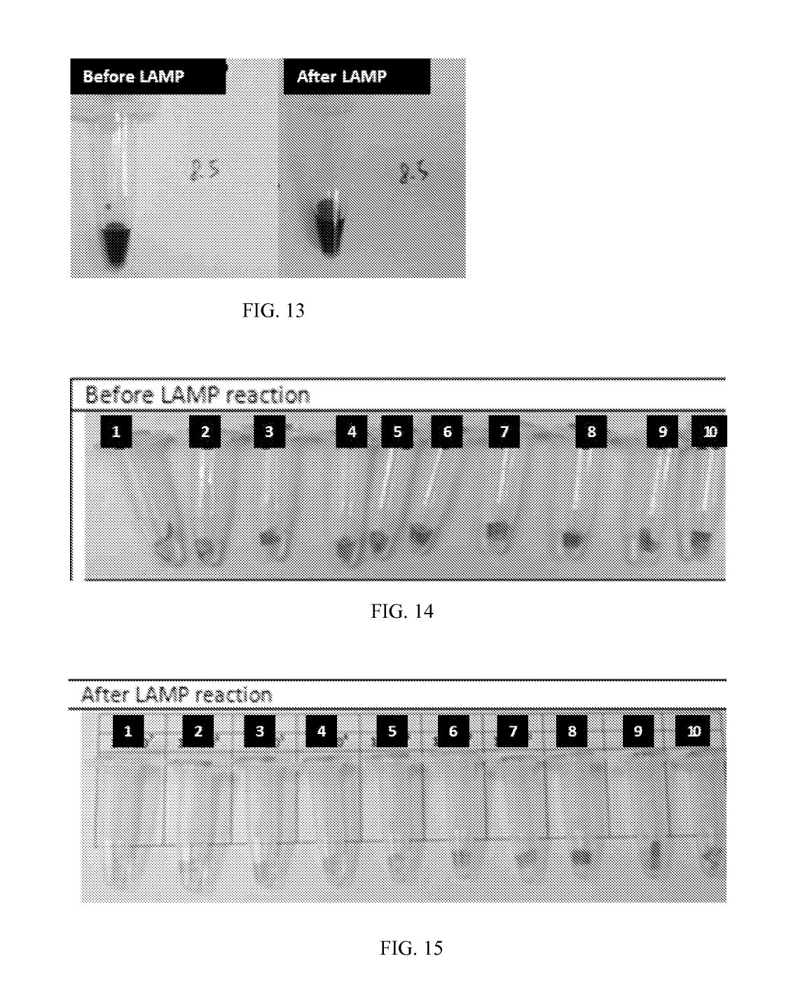

The bromothysial blue did not produce a colour change and the pH remains unchanged at 8.5 (See FIG. 13).

### Example 10

The reactions are set up to use lambda primer set (FIG. 22) and lambda genomic DNA. The DNA template is diluted into various concentration that represent from 1, 10, 100, 1,000, 10,000, 100,000, 1,000,000, and 10,000000 copies of lambda DNA. K2 film is included in each tube before the reaction. The negative control does not contain lambda DNA. The reaction is held at 63 degree Celsius for 30 mins and the colour of the reaction is observed. The K2 film changes colour from deep magenta to bright yellow when there is amplification. The limit of sensitive show in this assay is at 10 copies.

| | Final concentration | |
|---|---|---|
| Primers mix solution | | |
| Lambda_FIP | 1.6 | uM |
| Lambda_BIP | 1.6 | uM |
| Lambda_LF | 0.8 | uM |
| Lambda_LB | 0.8 | uM |
| Lambda_F3 | 0.2 | uM |
| Lambda_B3 | 0.2 | uM |
| LAMP buffer | | |
| KCl | 50 | mM |
| MgSO4 | 5 | mM |
| NH₄Cl | 5 | mM |
| BSA | 1 | mg/mL |
| Tween 20 | 0.10% | |
| Betain | 1M | |
| Deoxynucleotides | 2.8 | mM |
| Bst polymerase | 32.4 | U |

| | |
|---|---|
| H2O | Fill to 50 uL |

The dye colour changes to yellow for tubes 1 to 7. Tubes 8 to 10 remain pink. The result suggests the limit of detection is 10 coies of lambda DNA (See FIGS. 14 and 15).

TABLE 4

| | |
|---|---|
| Before reaction | After reaction |

| Tube | Template | pH | Colour | pH | Colour |
|---|---|---|---|---|---|
| number | Copies | value | value | value | value |
| 1 | 1 × 10⁷ | 8.16 | 5 | 6.30 | 1 |
| 2 | 1 × 10⁶ | 8.16 | 5 | 6.43 | 1 |
| 3 | 1 × 10⁵ | 8.16 | 5 | 6.65 | 1 |
| 4 | 1 × 10⁴ | 8.16 | 5 | 6.66 | 1 |
| 5 | 1 × 10³ | 8.16 | 5 | 6.85 | 1 |
| 6 | 1 × 10² | 8.16 | 5 | 6.86 | 1 |
| 7 | 1 × 10¹ | 8.16 | 5 | 6.88 | 1 |
| 8 | 1 | 8.16 | 5 | 7.30 | 4 |
| 9 | No template | 8.16 | 5 | 7.40 | 4 |
| 10 | No template | 8.16 | 5 | 7.49 | 4 |

The results of the colour value and the pH value of the reactions of differing copy numbers is provided.

The dye colour of each tube is pink before the LAMP reaction. Each tube corresponds to a lambda DNA concentration (See FIG. 14).

The dye colour changes to yellow for tubes 1 to 7. Tubes 8 to 10 remain pink. The result suggests the limit of detection is 10 coies of lambda DNA (See FIG. 15).

Table 5: The results of the colour value and the pH value of the re3actions of differing copy numbers is provided.

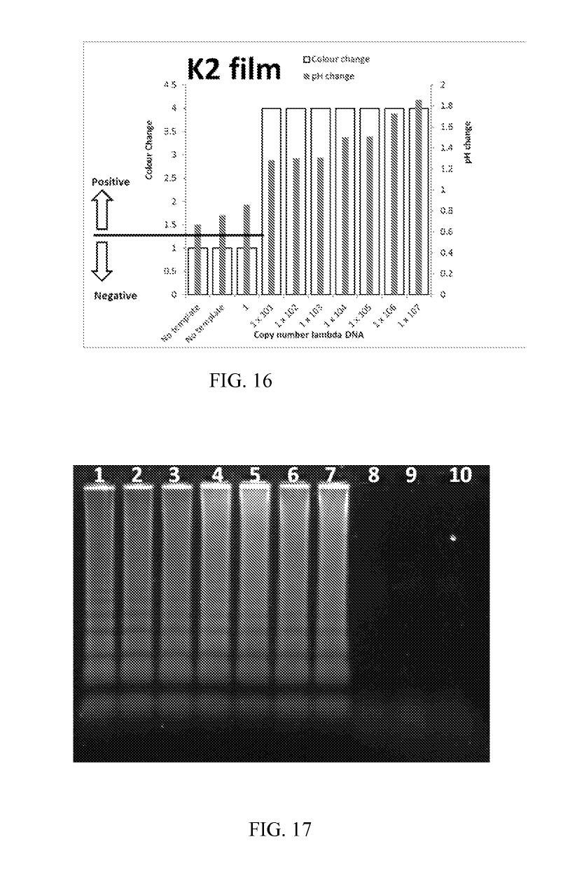

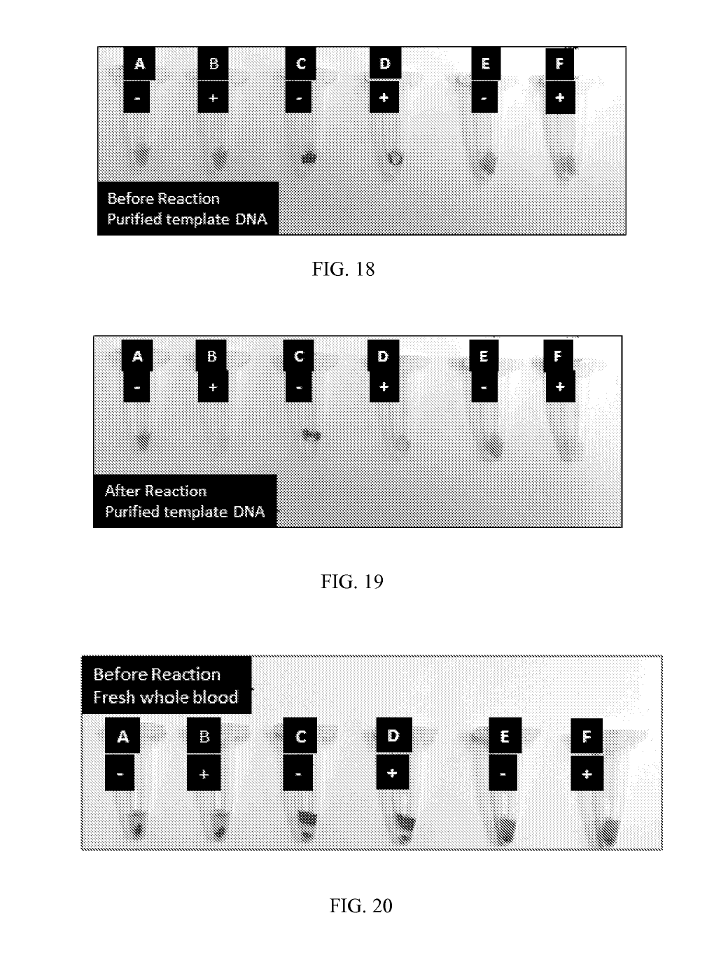

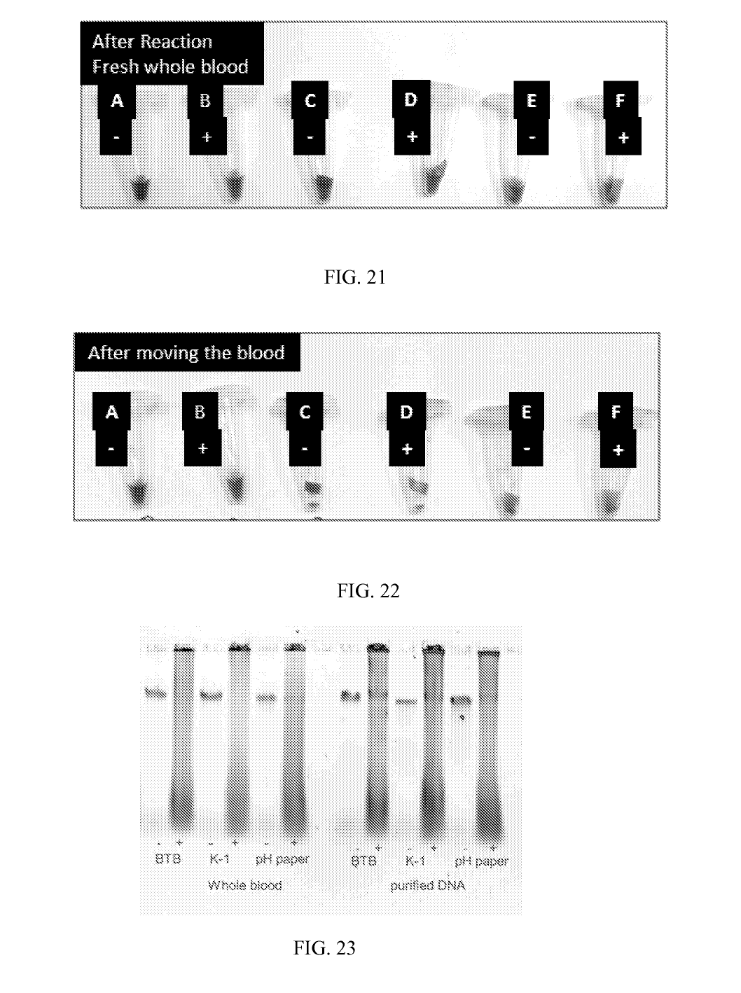

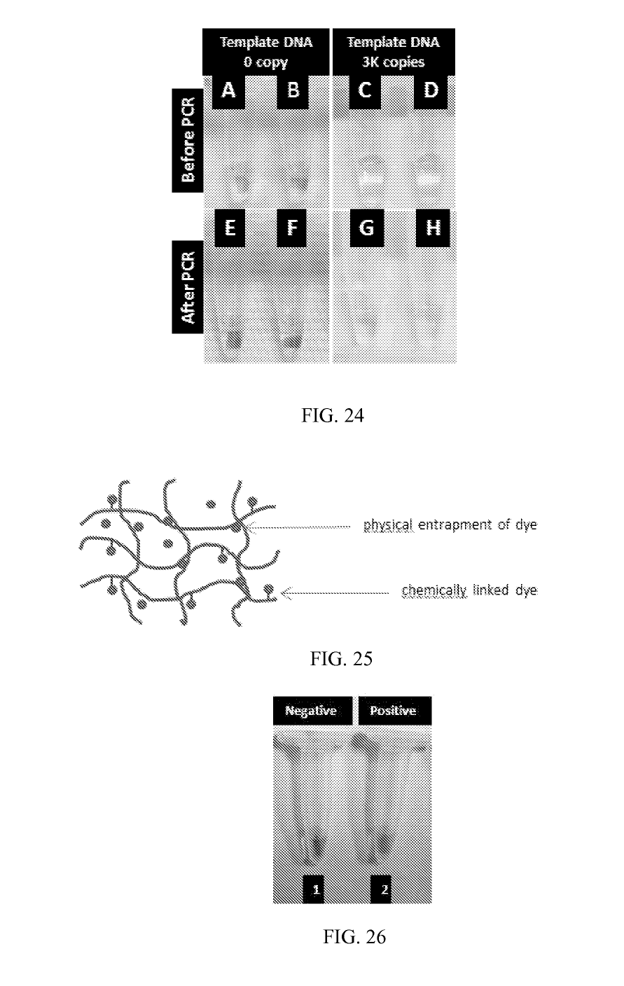

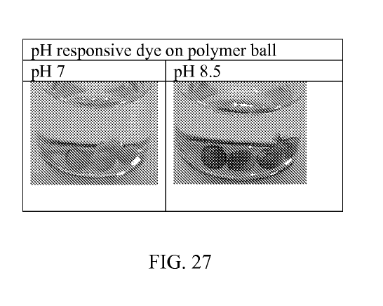

The chart shows the discrimination of positive and negative response is easily differentiated. The detection using K2 film shows as low as 10 copies of lambda DNA (See FIG. 16).

The agarose electrophoresis photo shows the LAMP amplification occurs with lane 1 to lane 7, where the copy number is 10,000,000, 1,000,000, 100,000, 10,000, 1,000, 100, and 10 respectively. Lane 8 is corresponding to a single copy of lambda DNA where there is not amplification observed. Lane 9 and 10 are reaction without lambda DNA.

### Example 11

In each tube, a soluble pH indicator (bromothymol blue, 0.1 mg/mL), K1 film and a pH testing paper (Merck Millipore cat#1.09543.0001, non-bleeding paper) is mixed with the amplification reagents before the LAMP reaction.

The reactions are set up to use p450 2C19 wild type primer set and K562 genomic DNA. 1 ng of K562 which is about 300 copies mixed with reaction components, 50 mM KCl, 5 mM MgSO4, 5 mM NH4Cl, 1 M betaine, 1 mg/mL BSA, 0.1% Tween 20, 2.8 mM dNTPs (deoxyadenosine triphosphate, deoxythymidine triphosphate, deoxyguanosine triphosphate, and deoxycytidine triphosphate), 1.6 microM FIP and BIP, 0.8 microM Loop-F and Loop-B, 0.2 microM F3 and B3, and 32U of Bst polymerase in 50 uL reaction. The pH is adjusted to 8.0 before adding Bst, K562, or whole blood) The dNTPs is replaced by a 2.8 mM mixture of (deoxyadenosine triphosphate, deoxyguanosine triphosphate, deoxycytidine triphosphate) in the negative control samples. In another panel, 2 micro litre of fresh whole blood from a finger prick is added into each tube. The reaction is held at 63 degree Celsius for 30 mins and the colour of reaction is observed.

It is very challenging to see the difference between amplification versus no amplification in the presence of the whole blood in all except the K1 film. As it is simple and easy to remove the cloudy whole blood solution from the K1 film, the nucleic acid amplification could be monitored as shown in the photos.

The photo shows the dye colour before the reactions that are with (positive) or without (negative) purified DNA. The reaction used purified DNA as the template. From the left to right, the tubes contain the dye: bromothymol blue (A and B), K1 film (C and D), and pH testing paper (E and F). At pH 8 the tube A and B are in light blue, tube C and D (K1 film) are in deep magenta, and tube E and F (pH paper from Merck-Millipore) are in greenish brown.

The photo shows the dye colour after the reaction that is with (positive) or without (negative) the DNA template. The colour of the dye changed when there was DNA template in the reaction. The tube B (bromothymol blue) changes from light blue to yellow. The tube D (K1 film) changes from deep magenta to orange. The tube F (pH paper) change from greenish brown to bright yellow.

The photo shows the whole blood effect on dye colour before the reactions. The tubes with template DNA are labelled with positive signed while the tubes without added DNA are labelled with negative sign. Each tube contains 2 microlitre of fresh whole blood. From the left to right, the tubes contain bromothymol blue (A and B), K1 film (C and D), and pH testing paper (E and F). At pH 8 the bromothymol blue is in light blue, K1 film is in deep magenta, and pH paper from Merck-Millipore is difficult to define the colour due to the heterogeneous colour mix.

The photo shows the whole blood effect after the reactions. The colour of the soluble dye, bromothymol blue (A and B), becomes indistinguishable with the presence of the whole blood.

The photo shows the colour of the immobilised dye after shaking the solution off the dye. The blood could be removed from the immobilised dye in the case of K1 film (C and D) and the pH paper (E and F). The removing process does not require user to open the tube therefore there is not risk of contamination. After removing the blood, the colour of the pH paper is also difficult to differentiate amplification (F) from no amplification (E). This is due to the porous structure of the paper that has trapped the blood within. The colour of K1 film is the only reaction that shows distinct difference between the no amplification (C, colour value=3) and amplification (D, colour value=1)

The LAMP reaction from each tube uses agarose electrophoresis. BTB is bromothymol blue (See FIG. 23).

### Example 12

### PCR Embodiment

| | Final concentration | |
|---|---|---|
| Primers mix solution | | |
| HCV core Forward primer | 1 | uM |
| HCV core Reverse primer | 1 | uM |
| PCR buffer | | |
| KCl | 50 | mM |
| MgCl4 | 2 | mM |
| Deoxynucleotides | 1 | mM |
| Taq polymerase | 2.5 | U |

| | |
|---|---|
| H2O | Fill to 30 uL |

The indicator dye film for monitoring the nucleic amplification is used in PCR. The film is compatible with the PCR reaction condition. In one example, the assay is assembled by using a plasmid containing a Hepatitis C virus core 1b gene. The reactions are setup with the dye film before the PCR reaction. The pH of each reaction is adjusted to between 8.0-8.2. The thermo-cycling programme follows an initial denaturation step at 94 degree Celsius for 2 minutes, with 55 repeats of three-step module: 94 degree Celsius for 30 seconds, 65 degree Celsius for 20 seconds, and 72 degree Celsius for 15 second. The reaction is finished holding the last step of the reaction at 72 degree Celsius for 2 minutes. The colour of the tubes is seen after they are taken out from the machine.

The result shows the distinct colour difference between tubes with amplification (yellow) and tubes without amplification (pink).

The result of the PCR reaction with the presence of the dye is provided in FIG. 33. K1 films are shown in A, C, E, and G while K2 films are shown in B, D, F, and H. Before the PCR reaction, all films show orange. After the PCR reaction, the tubes without amplification (E and F) show pink. The tubes with plasmid templates where the amplification occurs show yellow (G and H).

### Example 13

It has been a long felt wish for the development of an assay that could detect a gene without sample preparation and without requiring more than 2 steps from sample to end result and without instruments for the result interpretation. The present invention provides a method that fulfills these requirements. Genes are amplified in the presence of whole blood directly from the finger prick. The presence of the gene is detected by monitoring the amplification using an immobilised dye. The results in reducing all these steps into one.

First, water is loaded from a predefined volume container to one or more reaction container(s) that contain lyophilised amplification reagents in the presence of the indicator dye and the sample is loaded, such as whole blood, into the reaction container(s).

To prevent contamination, the container should remain instrument remain securely closed after any nucleic acid amplification. Without the help of any instruments, the amplification result would usually be difficult to read, when the amplification reaction is not a clear solution, such as whole blood amplification. To overcome the interference from the suspended colloidal particles or the coloured compounds that come with the sample, the samples are usually pre-treated by dilution or heating or both. Examples cover the conventional detection without instrument such as DNA chelating fluorescence dye, YO-PRO-1 or Sybr Green (Genome Letters, 2, 119-126, 2003), metal chelating dye, Calcein and hydroxy naphthol blue (Biotechniques, 46, 167-172, 2009).

The present invention demonstrates that the dye chemicals (K1 and K2) are covalently linked to a hydrogel 3D object which fits into the container where the amplification occurs. It is shown from our disclosure using films that are conjugated with the K1 or K2, allow the unaided eyes to easily read the nucleic acid amplification result. However, without opening the reaction container, it is not always easy to separate the solution from the film in a container, as the film tends to stick to the wall of the container. The 3D object solves the problem by minimizing the contact surface between the indicator dye and the container.

The 3D object is a ball such that the contact area between the 3D object and the reaction is minimized. The 3D ball can be formed by applying a layer of hydrogel to a ball, such as polystyrene ball, cellulose ball, or ball made of other material. Different colours of the ball are selected to enhance the contrast of the indicator colour dye to facilitate even better colour change for the unaided eye.

The present invention also describes a design where the dye is an indicator ball or a 3D dye indicator object is influenced by an external magnetic field. When paramagnetic or ferromagnetic material is embedded in the 3D object or ball, it is possible to control the position of the dye such that the dye can be viewed without the interference of the cloudy solution and is done so with the container securely sealed. The embedding is as simple as punching an iron pin into a polymer ball before the hyrogel coating.

Yet in another embodiment, the 3D object is a collection of small particles that can form a cluster of 3D objects under the influence of an external magnetic force. The particles are of micro meter in diameter in equivalent to a spherical ball or other sizes that are reasonably easy for magnetic manipulation.

The hydrogel is made up of Poly(2-hydroxyethyl methacrylate) (PHEMA), Polyurethane (PU), Poly(ethylene glycol) (PEG), polyethylene glycol methacrylate (PEGMA), polyethylene glycol dimethacrylate (PEGDMA), polyethylene glycol diacrylate (PEGDA), Poly (vinyl alcohol) (PVA), Poly(vinyl pyrrolidone) (PVP), or Polyimide (PI).

The dye is any reactive vinylsulphonyl dye or pH indicator dye.

A hydrogel is formed by using poly(2-hydroxyethyl methacrylate), the hydrogel is conjugated with K2 dye, also known as 4-[4-(2-Hydroxyethanesulfonyl)-phenylazo]-2,6-dimethoxyphenol indicator dye (vinylsulphonyl dye)

### The Material are:

1) 2-hydroxyethyl methacrylate (HEMA), poly(ethylene glycol) dimethacrylate, 2,2-Dimethoxy-2-phenylacetophenone, 4-[4-(2-Hydroxyethanesulfonyl)-phenylazo]-2,6-dimethoxyphenol (pH indicator dye), Sulfuric acid, Sodium hydroxide, and Sodium carbonate

### Hydrogel Preparation

The Chemical composition of reagents used in the hydrogel are given in table 6.

TABLE 6
Chemical composition of reagents used for formation of hydrogel.

| Reagent | Mass % |
|---|---|
| HEMA | 63 |
| poly(ethylene glycol) dimethacrylate | 1.5 |
| 2,2-Dimethoxy-2- | 0.5 |
| phenylacetophenone (DMPA) | |
| DI water | 35 |

Table 5: Chemical composition of reagents used for formation of hydrogel

All the reagents are added together after weighing and subjected to stirring for 10 min to obtain a homogeneous mixture. This mixture is solvent casted into the glass petridish. The petridish is subjected to UV irradiation for 3 min where both, polymerization and cross-linking reaction is carried out. Under UV, dissociation of DMPA (photo initiator) takes place, generating two radicals for each photo initiator molecule. The radicals initiate polymerization of HEMA to form PHEMA and simultaneously poly(ethylene glycol) dimethacrylate (cross linker) is also activated to carry out intermolecular cross-linking of PHEMA chains. After 3 min, hydrogel is delaminated from petridish and dipped into DI water for 1 hr to ensure removal of all the by-products and unreacted reagents.

### Chemical Staining of PHEMA Hydrogel with 4-[4-(2-Hydroxyethanesulfonyl)-phenylazo]-2,6-dimethoxyphenol

In a typical immobilisation procedure, 100 mg of the indicator dye is thoroughly mixed (in a mortar with a pestle) with 1 g concentrated sulfuric acid and left for 30 min at room temperature. This converts the 2-hydroxyethylsulfonyl group of indicator dye into the sulfonate. The mixture is then poured into 900 ml of distilled water and neutralised with 1.6 ml 32% sodium hydroxide solution. Then, 25.0 g of sodium carbonate dissolved in 100 ml water and subsequently, 5.3 ml of 32% sodium hydroxide solution are added. At this stage, PHEMA hydrogel layers are placed into this dyeing solution. Under basic conditions, dye sulfonate is converted into the chemically reactive vinylsulfonyl derivative, and simultaneously, Michael addition of the vinylsulfonyl group with reactive groups of the polymer, (e.g. the hydroxyl groups of the PHEMA hydrogel) takes place. After 12 h, the coloured layers are removed from the dyeing bath and washed several times with distilled water.

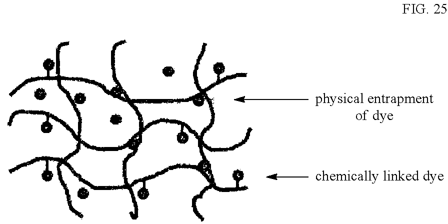

At this stage, the dye molecule is chemically linked to the cross-linked polymer matrix. Also due to hydrogel's ability to absorb aqueous solutions, the dye gets physically loaded into the matrix. This is non-covalent type of binding of dye to the polymer as shown herein below. After enough washing, leaching of dye from the hydrogel is stopped, and at this stage coloured hydrogel is cut into small pieces to be used in nucleic acid testing.

### Example 14

The reactions are set up to use lambda primer set and lambda DNA. About 10 billion copies lambda DNA are mixed with reaction components with the presence of a slab of hydrogel (tube 2). The dNTPs are replaced by a 2.8 mM mixture of (deoxyadenosine triphosphate, deoxyguanosine triphosphate, deoxycytidine triphosphate) in the negative control sample (tube 1). The reaction is held at 63 degree Celsius for 30 mins, and the colour of reaction is observed. The hydrogel slab is about 2 mm×4 mm×1 mm. At the end of the reaction, it is clear that the hydrogel slab changes from magenta to orange with the presence of all four deoxynucleotides, while the colour remains magenta when the missing deoxythymidine triphosphate prevented LAMP reaction.

| | Final concentration | |
|---|---|---|
| Primers mix solution | | |
| Lambda_FIP | 1.6 | uM |
| Lambda_BIP | 1.6 | uM |
| Lambda_LF | 0.8 | uM |
| Lambda_LB | 0.8 | uM |
| Lambda_ F3 | 0.2 | uM |
| Lambda_B3 | 0.2 | uM |
| LAMP buffer | | |
| KCl | 50 | mM |
| MgSO4 | 5 | mM |
| NH₄Cl | 5 | mM |
| BSA | 1 | mg/mL |
| Tween 20 | 0.10% | |
| Betain | 1M | |
| Deoxynucleotides | 2.8 | mM |
| Bst polymerase | 32 | U |

| | |
|---|---|
| H2O | Fill to 50 uL |

The colour difference between the reaction versus no reaction when hydrogel slabs are used is provided in FIG. 26.

| | | Before | After |
|---|---|---|---|
| | | LAMP | LAMP |
| K2 | Lamp | Colour | Colour |
| hydrogel | reaction | value | value |
| Tube 1 | No | 5 | 5 |
| Tube 2 | Yes | 5 | 3 |

Example of pH responsive dye conjugated hydrogel of polyurethane on a cellulose acetate ball of 2 mm diameter.

The pH response of the core-shell hydrogel particles. The hydrogel coated cellulose acetate is covalently linked with the pH indicator dye, and the colour of the dye is displayed. At pH 7, the colour is yellow. At pH 8.5, the colour is magenta.

### Lambda Primer set

Lambda_FIP
5′-CAGCATCCCTTTCGGCATACCAGGTGGCAAGGGTAATGAGG-3′
Lambda_BIP
5′-GGAGGTTGAAGAACTGCGGCAGTCGATGGCGTTCGTACTC-3′
Lambda_F3
5′-GAATGCCCGTTCTGCGAG-3′
Lambda_B3
5′-TTCAGTTCCTGTGCGTCG-3′
Lambda_LF
5′-GGCGGCAGAGTCATAAAGCA-3′
Lambda_LB
5′-GGCAGATCTCCAGCCAGGAACTA-3′

### CYP2C19 Primer Set

2C19_F3
5′-CCA GAG CTT GGC ATA TTG TAT C-3′
2C19_B3
5′-AGG GTT GTT GAT GTC CAT-3′
2C19_FIP.Wild
5′-CCG GGA AAT AAT CTT TTA ATT TAA ATT ATT GTT
TTC TCT AG-3′
2C19_BIP.Wild
5′-CGG GAA CCC GTG TTC TTT TAC TTT CTC C-3′
2C19_FIP.Mut
5′-CTG GGA AAT AAT CTT TTA ATT TAA ATT ATT GTT
TTC TCT AG-3′
2C19_BIP.Mut
5′-CAG GAA CCC GTG TTC TTT TAC TTT CTC C-3′
2C19_LF
5′-GAT AGT GGG AAA ATT ATT GC-3′
2C19_LB
5′-CAA ATT ACT TAA AAA CCT TGC TT-3′

Primer sequence:

| | |
|---|---|
| HCV core Forward primer | GTCGCGTAACTTGGGTAAGG |
| HCV core Reverse primer | AAGCTGGGATGGTCAAACAG |

### Example 15

A device wherein the sensors and detection circuits are electronically printed and used is made. A pH sensor is built that has three layers. One is the layer that houses the substrate or analyte to be tested. It also contains at least two electrodes, each covered by a pH sensing material. In this example, polyaniline is used. Finally, there is a third layer that acts as an insulator in order to place a barrier between the electrodes and the substrate or analyte.

A resistor and battery are also part of the system or device.

In order to measure the resistance change being measured in this example, a divider circuit is used to have the value measured as a change in voltage.

This device is used to measure a pH level particularly used. If a need is such that a pH range needs to be measured, more than one divider circuit is used. Furthermore, various readings in time are taken for a potentially linear or other response to be measured.

### Example 16

Polyaniline is used as a film covering two planar photoconductors. The polyaniline film acts as a color control filter. One of the photoconductors of this device acts as a control, and its polyaniline film does not contact the analyte being measured for pH change. The other photoconductor is the testing portion of the device, and its polyaniline film does react with the analyte and changes color based on the pH response. A voltage divider for a battery is also provided. The polyaniline is green at acidic pHs and blue at basic pHs.

1. A medical testing device, said device comprising: printed electronic circuits, display and battery, a sensor, a monitor, a reading and display unit, wherein at least one of the electronics is printed or wherein said device uses colorimetric media to detect changes in color in measuring chemical or biological reactions.

2-87. (canceled)

88. The device according to claim 1, wherein the device is an integrated testing lateral flow device, and wherein the electronics are printed using organic semiconductor materials.

89. The integrated testing lateral flow device according to claim 88, wherein the organic semiconductor materials are poly(3-hexylthiophene), pentacene, polytriarylamine, 5′,5-bis-(7-dodecyl-9H-fluoren-2-yl)-2,2′-bithiopene, polyethylene, naphthalate, poly(4,4′ didecylbithiopene-co-2,5-thieno[2,3-b] thiophene, polyaniline or combinations thereof.

90. The integrated testing lateral flow device according to claim 89, wherein the electronics are printed using inorganic materials, and wherein said inorganic materials are tantalum peroxide, silver chloride, silver paste, silicon, silicon dioxide, silicon nitride, aluminum oxide, mineral semiconductors, metals, metal oxides or combinations thereof.

91. A diagnostic kit for a device that contains a chromatographic medium, said device comprising: (i) a sample loading zone located upstream of a detection zone; (ii) a reporting carrier zone located between the sample loading zone and a detection zone, wherein the reporting carrier zone comprises a reporting carrier capable of forming a complex with an analyte and wherein said device contains said reporting carrier, a carrier and one or more proficient enzyme cassettes; and (iii) a detection zone, wherein said detection zone contains a capture component for the analyte and an indicator.

92. The kit according to claim 91 additionally comprising: a sample load zone with test sample; wherein the test sample travels through the reporting carrier zone along the chromatographic medium from the sample loading zone to the detection zone and beyond the detection zone, a sample load zone with test sample; wherein the test sample is mixed with the reporting carrier before loading a sample load zone, adding a substrate to the detection zone; and wherein the substrate undergoes a reaction in the presence of proficient enzyme analyte containing reporting carrier and generating a response of the indicator within the detection zone that corresponds to the presence or absence of the analyte in the test sample.

93. A diagnostic kit to detect the presence of an analyte using an enzyme-aided amplification method in a chromatographic medium, said kit comprising: an analyte as a biomarker, an entity to recognize said biomarker; a reporting carrier that has a first entity which binds to the analyte, and a capture component, wherein said biomarker is an antigen, said capture component is a second antibody that binds to the analyte at a different epitope from the first antibody.

94. The diagnostic kit according to claim 93; wherein the first antibody is covalently cross-lined to A proficient enzyme, wherein the reporting carrier comprises streptavidin and biotinylated proficient enzyme and biotinylated antibody; and wherein the first antibody is associated with the proficient enzyme through non-covalent Streptavidin-Biotin interaction.

95. The diagnostic kit according to claim 94, wherein the analyte is a sequence of nucleic acids.

96. The diagnostic kit according to claim 95, wherein the reporting carrier comprises a first sequence of nucleic acids which hybridise to one part of the target nucleic acid sequence, and wherein the proficient enzyme which is associated with the first nucleic acid.

97. The diagnostic kit according to claim 96, wherein the first nucleic acid is covalently cross-linked to the proficient enzyme.

98. The diagnostic kit according to claim 97, wherein the reporting carrier comprises: streptavidin and biotinylated proficient enzyme and biotinylated first nucleic acid, and wherein the first nucleic acid is associated to the proficient enzyme through non-covalent Streptavidin-Biotin interaction.

99. The diagnostic kit of claim 93, wherein the reporting carrier binds to the p24 protein of HIV, HBV, HCV, HPV, or herpes virus; the nucleic acid or proteins of bacterial proteins for syphilis, chlamydia, gonorhea; lipoproteins or the nucleic acids thereof; glycoproteins or the nucleic acids thereof.

100. The diagnostic kit of claim 95, wherein the reporting carrier binds to HIV nucleic acid.

101. A method for measuring a chemical or biological reaction, said method comprising: using a printed electronic device containing at least one of the electronics selected from the group circuits, display, battery, sensor, monitor, reading unit, display unit, adding of an analyte; observing the reaction; reading the result; that has a data input and data output mechanism and a power input mechanisim.

102. The method according to claim 101, wherein the device is a lateral flow device or microfluidic device.

103. A device according to claim 1, wherein the device uses colorimetric media to detect changes in color or to visualize chemical or biological reactions; wherein the colorimetric medium is a pH sensitive indicator dye; and wherein the pH sensitive dye is in solution; immobilized on one or more 3D structure; immobilized in a reaction chamber or vessel; or combinations thereof.

104. A method for testing for pH changes, said method comprising: using the device of claim 103 to measure said pH change.

105. The device according to claim 103, wherein the pH sensitive dye is immobilized on one or more 3D structures; wherein the pH sensitive dye is immobilized in a reaction chamber or vessel; and wherein a combination of a pH sensitive dye is immobilized in solution, or one or more 3D structures or in a reaction chamber or vessel.

106. The device according to claim 1, wherein the printed electronics are nanoparticles, nanotubes, graphene or combinations thereof, and wherein the printed electronics are printed by atomic layer deposition, vapour deposition, inject printing, roll-to-roll printing, screen printing or combinations thereof.

107. The device according to claim 1, additionally comprising: transistor, controlling circuits, signal circuit, display circuit, battery, input and output for data recording and power sourcing.

108. A device for measuring pH changes, wherein said device comprises: a printed sensor system containing a test line, a control line and a conjugation pad.

109. The device according to claim 108, further comprising an absorption pad.

110. The device according to claim 108, wherein said device provides Yes/No semi quantitative, or quantitative display; a line appearance where no line was previously read; a line appearance on a white background; the appearance of different pattern; a color change; the disappearance of a line; or combinations thereof.

111. The device according to claim 108, wherein at least one light emitting diode or an array of light emitting diodes with text information related to the assay result is displayed.

112. A medical testing device, said device comprising: a printed battery, a printed sensor and a communication module to upload information, wherein said communication module uploads information to a base station.

113. An integrated testing lateral flow device comprising a chromatographic medium having a sample loading zone upstream from a detection zone; a reporting carrier zone; a detection zone; wherein the reporting carrier is located between the sample loading zone and the detection zone.

114. The device according to claim 113, wherein the reporting carrier comprises a carrier and one or more proficient enzymes; wherein the presence or absence of an analyte is detected, by an enzyme-aided amplification method in a chromatographic medium; and wherein the analyte is a biomarker antigen that is recognized by an antibody.

115. The device according to claim 114, wherein the reporting carrier comprises a first antibody that binds to the analyte and proficient enzyme; and the capture component comprises a second antibody that binds to the analyte at a different epitope from the first antibody.

116. The device according to claim 115, wherein the reporting carrier is streptovidin, biotinylated proficient enzyme, biotinylated antibody and/or combinations thereof, and wherein the analyte is a nucleic acid sequence.

117. The device according to claim 114, wherein the reporting carrier binds to HIV nucleic acids.

118. The device according to claim 93, comprising: an indicator dye that is potassium 1-hydroxy-4-[4-(hydroxyethylsulphonyl)-phenylazo]-naphthalene-2-sulphonate or 4-[4-(2-hydroxyethanesulfonyl)-phenylazo]-2,6-dimethoxyphenol or any reactive vinylsulphonyl dye or combinations thereof.

119. The device according to claim 93, wherein the indicator dye is mixed in the reaction reagents.

120. The device according to claim 93, wherein the pH indicator dye is part of the amplification reagent prior to the reaction.

121. The device according to claim 93, wherein the pH indicator dye is added after the reaction.

122. The device according to claim 93, wherein the pH indicator is immobilized to thin particles microparticles, a thin film, or a three-dimensional object.

123. The device according to claim 122, wherein the particles are micro particles made of polymer, porous particles, or core-shell particles; and wherein the dye is covalently conjugated to the micro-particles, wherein the particles are made of polymer, porous particles or core-shell particles, and wherein one or more particles are viable as discrete particles, combining at least one or more particles.

124. The device according to claim 122, wherein the three dimensional object is under the influence of external magnetic force; wherein the thin film is a film covalently conjugated with the pH indicator dye; and wherein the three-dimensional object is made of hydrogel or is coated on the surface of non-hydrogel three-dimensional object of milli or micro meter size.

125. The device according to claim 122, wherein the three-dimensional object is mixed with the non-hydrogel material to increase the mass density to enhance the colour intensity introduction of coloured background from the non-hydrogen material.

126. The device according to claim 122, wherein the three-dimensional object is a collection of small particles that form a cluster of three-dimensional objects under the influence of an external magnetic force.

127. The device according to claim 126, wherein the three-dimensional object is one or more milli particles and wherein the milli particles are moved within the reaction container under the influence of an external magnetic force.

128. The device according to claim 122, wherein the hydrogel is made of Poly(2-hydroxyethyl methacrylate) (PHEMA), Polyurethane (PU), Poly(ethylene glycol) (PEG), polyethylene glycol methacrylate (PEGMA), polyethylene glycol dimethacrylate (PEGDMA), polyethylene glycol diacrylate (PEGDA), Poly (vinyl alcohol) (PVA), Poly(vinyl pyrrolidone) (PVP), or Polyimide (PI), or combinations thereof.

129. The device according to claim 93, wherein one or more indicator dye works as an indicator for the starting pH.

130. The device according to claim 129, wherein one or more pH indicators is used with each pH indicator being subject to a different pKa; wherein at least two pH indicators are used to give an indication that the starting pH is out of range, wherein the presence of a line or other pattern where no line existed prior to the reaction indicates an observed chemical or biological reaction, and wherein a colorimetric change indicates the observation of chemical or biological reaction.

131. The device according to claim 93, wherein an amplification method is used to carry out the reaction and the method is a thermocycling method or an isothermal method.

132. The method according to claim 131, wherein the thermocycling method is PCR, real-time PCR or reverse transcription PCR, wherein the isothermal method is a Loop-mediated Amplification (LAMP), Strand Displacement Amplification (SDA), Recombinanse Polymerase Amplification (RPA), Nucelic Acid Sequence-Based Amplification (NASBA), Transcription-Mediated Amplification (TMA), SMART (Nucl. Acids Res. 29:e54, 2001), Helicase-Dependent Amplification (HDA), Cross Priming Amplification (CPA), Rolling-Circle Amplification (RCA), ramified rolling circle amplification (RAM), Nicking enzyme amplification reaction (NEAR), Nicking Enzyme Mediated Amplification (NEMA, CN100489112 C), Isothermal Chain Amplification (ICA), Exponential Amplification Reaction (EXPAR), Beacon-Assisted Detection Amplification (BAD AMP) Primer Generation-Rolling Circle Amplification (PG-RCA), or other nucleic acid amplification methods, wherein the amplification does not require thermal cycling.

133. A kit to detect nucleic acid amplifications, said kit comprising: (a) one or more container(s), (b) amplification reagents, and (c) at least one pH indicator, wherein the pH indicator is potassium 1-hydroxyl-4-[4-(hydroxyethyl sulphonyl)-phenylazo]-naphthalene-2-sulphonate or 4-[4-(2-hydroxylethanesulfonyl)-phenylazo]-2,6-dimethoxyphenol or any reactive vinylsulphonyl due or combination thereof.

134. A pH detection device comprising: a pH sensitive sensor component controlling circuitry for calculating signal strength and display pixel components, wherein said components are printed on dielectric materials, wherein the dielectric material is flexible plastic.

135. A device for detecting colorimetric changes in a chemical or biological sample, said device comprising:
a. at least two photo conductors electronically printed on electrodes;
b. an organic film covering one of the photoconductor, wherein the one film covering one of the photoconductors is acting as a control and does not interact with a test medium;
c. a pH measuring device that detects color change of the other photoconductor;
d. a battery; and
e. data and power input and output.

136. The device according to claim 135, wherein an organic material used as the film is polyalanine and wherein a pH change is reflected as a color change.

137. A device for measuring a pH change by using printed electronics that measures conductivety according to a pH change, said device comprising:
a. a compartment or layer wherein at least two electrodes are placed and wherein the electrodes are printed electrodes and wherein a substrate or analyte is placed;
b. a second compartment or layer thereafter that contains printed pH sensing materials; and
c. a third compartment or layer that has insulation to cause a barrier between the printed electrodes and the substrate or analyte.

138. The device according to claim 137, additionally comprising a divider circuit to record a resistance into a measurable voltage, wherein the voltage change is displayed electrophoretically and/or electrochromatically.

139. A method for sensing a chemical and/or biological reaction, said method comprising:
a. detecting an electrical signal output;
b. monitoring the electrical signal when the pH changes in the chemical and/or biological reaction; wherein at least one of the detection and monitoring components for electronically detection of the reaction is on a substance.

140. The method according to claim 139, wherein the detection of said chemical and/or biological reaction is in a single zone; using differential output; or measuring pH at different time points during the reaction, wherein the reaction is a PCR reaction, real-time PCR or revise transcription PCR, and wherein the reaction is a colorimetric reaction.

141. The method according to claim 139, wherein the reaction is an isothermal reaction.

142. The method according to claim 141, wherein the isothermal reaction is a single stranded displacement amplification (SDA), DNA amplification, RNA amplification, or combinations thereof.

143. The method according to claim 93, wherein the sensitive indicator dye is lyophilised with the amplification reagent; and wherein at least two pH indicators are used to give an indication that the starting pH is out of range.

144. The method according to claim 143, wherein each sensitive indicator dye works as an indicator for the starting pH.

145. The method according to claim 140, wherein the isothermal method of a Loop-mediated Amplification (LAMO), Strand Displacement Amplification (SDA), Recombinase Polymerase Amplification (RPA), Nucleic Acid Sequence-Based Amplification (NASBA), Transcription-Mediated Amplification (TMA), SMART (Nucl. Acids Res. 29:e54, 2001), Helicase-Dependent Amplification (HDA), Cross Priming Amplification (CPA), Rolling-Circle Amplification (RCA), ramified rolling circle amplification (RAM), Nicking enzyme amplification reaction (NEAR), Nicking Enzyme Mediated Amplification (NEMA, CNio0489112 C), Isothermal Chain Amplification (ICA), Exponential Amplification Reaction (EXPAR), Beacon-Assisted Detection Amplification (BAD AMP), Primer Generation-Rolling Circle Amplification (PG-RCA), or other nucleic acid amplification methods, wherein the amplification does not require thermal cycling.
